(19) United States
(12) Patent Application Publication
(10) Pub. No.: US 2009/0318349 A1
(43) Pub. Date: Dec. 24, 2009

(54) TRANSGLUTAMINASE VARIANTS WITH IMPROVED SPECIFICITY

(75) Inventors: Zhixiang Hu, The Woodlands, TX (US); Xin Zhao, Beijing (CN); Jianhua Wang, Beijing (CN); Chih-Chuan Chang, Bangkok (TH); Leif Norskov-Lauritsen, Tappernoje (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk Health Care AG, Zurich (CH)

(21) Appl. No.: 12/310,275

(22) PCT Filed: Aug. 17, 2007
(86) PCT No.: PCT/EP2007/058571

§ 371 Date: Mar. 26, 2009

(30) Foreign Application Priority Data
Aug. 18, 2006 (EP) PCT/EP2006/065439
Feb. 22, 2007 (EP) 07102885.6
Feb. 22, 2007 (EP) 07102886.4

(51) Int. Cl. A61K 38/27; C12N 9/00; C07K 14/195; C07H 21/00; C12N 15/63; C07K 14/61; C12N 1/19; C12N 1/15; C12N 1/21

(52) U.S. Cl. 514/12; 435/183; 530/350; 536/23.7; 435/320.1; 530/399; 435/254.2; 435/254.11; 435/252.3

(57) ABSTRACT

Variants of transglutaminase from Streptoverticillium ladakanum, which variants have improved selectivity for Gln-141 of human growth hormone are provided.

### FIELD OF THE INVENTION

The present invention relates to novel variants of transglutaminase from Streptoverticillium ladakanum. The variants may be used for site-specific modification of peptides at designated glutamine residues with improved selectivity.

### BACKGROUND OF THE INVENTION

It is well-known to modify the properties and characteristics of peptides by conjugating groups to said proteins which duly changes the properties. In particular for therapeutic peptides it may desirable or even necessary to conjugate groups to said peptides which prolong the half life of the peptides. Typically such conjugating groups are polyethylene glycol (PEG), dextran, or fatty acids—see J. Biol. Chem. 271, 21969-21977 (1996).

Transglutaminase (TGase) has previously been used to alter the properties of peptides. In the food industry and particular in the diary industry many techniques are available to e.g. cross-bind peptides using TGase. Other documents disclose the use of TGase to alter the properties of physiologically active peptides. EP 950665, EP 785276 and Sato, Adv. Drug Delivery Rev. 54, 487-504 (2002) disclose the direct reaction between peptides comprising at least one Gln and amine-functionalised PEG or similar ligands in the presence of TGase, and Wada in Biotech. Lett. 23, 1367-1372 (2001) discloses the direct conjugation of β-lactoglobulin with fatty acids by means of TGase, and Valdivia in J. Biotechnol. 122, 326-333 (2006) reported TGase catalyzed site-specific glycosidation of catalase. WO2005070468 discloses that TGase may be used to incorporate a functional group into a glutamine containing peptide to form a functionalised peptide, and that this functionalised peptide in a subsequent step may be reacted with e.g. a PEG capable of reacting with said functionalised protein to form a PEGylated peptide.

Transglutaminase (E.C.2.3.2.13) is also known as protein-glutamine-γ-glutamyltransferase and catalyses the general reaction

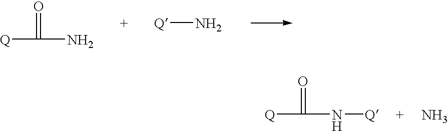

wherein Q-C(O)—NH₂ may represent a glutamine containing peptide and Q′-NH₂ then represents an amine donor providing the functional group to be incorporated in the peptide in the reaction discussed above.

A common amine donor in vivo is peptide bound lysine, and the above reaction then affords cross-bonding of peptides. The coagulation factor Factor XIII is a transglutaminase which effects clotting of blood upon injuries. Different TGases differ from each other, e.g. in what amino acid residues around the Gln are required for the protein to be a substrate, i.e. different TGase's will have different Gln-containing peptides as substrates depending on what amino acid residues are neighbours to the Gln residue. This aspect can be exploited if a peptide to be modified contains more than one Gln residue. If it is desired to selectively conjugate the peptide only at some of the Gln residues present this selectivity can be obtained be selection of a TGase which only accepts the relevant Gln residue(s) as substrate.

Human growth hormone (hGH) comprises 13 glutamine residues, and any TGase mediated conjugation of hGH is thus potentially hampered by a low selectivity. It has previously been described that out of 13 glutamine (Gln) residues on hGH, two (Q141 and Q40) glutamines are reactive under the catalysis of TGase (WO2006/134148). There is a need for identifying TGases, which mediates a still more specific functionalization of hGH.

### SUMMARY OF THE INVENTION

It has now been determined, that mTGase (the term mTGase is used for denoting a TGase as expressed by the microbial organism from which it is isolated) from Streptoverticillium ladakanum (the mTGase from S. ladakanum may be abbreviated as mTGase-SL) has even higher site-specificity (also called selectivity), doubled that of the mTGase of Streptomyces mobaraensis. It has also been found that added residues at the N-terminus of the mTGase, for instance as a result of different expression and processing strategies or as deliberate mutations, enhances such site-specificity. For instance, if the selectivity of mTGase from S. mobaraensis for Gln141 over Gln40 is set to be 1, native, Met-, AlaPro- or GlyPro-mTGase from S. ladakanum gives a relative selectivity of 1.7; 1.8; 2.1 and 2.7, respectively.

The present invention provides a transglutaminase peptide having a specificity for Gln-141 of hGH compared to Gln-40 of hGH, which is higher than the specificity of a peptide having an amino acid sequence as shown in SEQ ID No. 1 for Gln-141 of hGH compared to Gln-40 of hGH.

In one embodiment, the invention relates to an isolated peptide comprising an amino acid sequence having at least 80% identity with the amino acid sequence in SEQ ID No. 1, wherein said sequence is modified in one or more of the positions to the amino acid residues Tyr62, Tyr75 and Ser250 of SEQ ID No. 1.

In one embodiment, the invention relates to an isolated peptide comprising an amino acid sequence having at least 80% identity with the amino acid sequence in SEQ ID No. 1, wherein said sequence is modified by the addition of from one to ten amino acids in the N-terminal.

In one embodiment, the invention relates to a nucleic acid construct encoding a peptide according to the present invention.

In one embodiment, the invention relates to a vector comprising a nucleic acid encoding a peptide according to the present invention.

In one embodiment, the invention relates to a host comprising a vector comprising a nucleic acid encoding a peptide according to the present invention.

In one embodiment, the invention relates to a composition comprising a peptide according to the present invention.

In one embodiment, the invention relates to a method of conjugating hGH, the method comprising reacting hGH with an amine donor in the presence of a peptide according to the present invention.

### BRIEF DESCRIPTION OF THE FIGURES

FIG. 1 shows a sequence alignment of the sequence of the mTGase from Streptomyces mobaraensis and the mTGase from Streptoverticillium ladakanum.

FIG. 2A Blank for the reaction: wild type hGH with 1,3-dimaninol propanol. No mTGase was added.

FIG. 2B. AlaPro-mTGase from S. mobaraensis. Reaction time=30 minutes; Selectivity=5.7; conversion rate=32%

FIG. 2C. GlyPro-mTGase-SL; Reaction time=15 m; Selectivity=10.31; hGH conversion rate=55%.

FIG. 2D. GlyPro-mTGase_Y75A-SL; Reaction time=300 m; Selectivity=17.33; hGH conversion rate=40%.

FIG. 2E. GlyPro-mTGase_Y75F-SL; Reaction time=75 m; Selectivity=20.94; hGH conversion rate=33%.

FIG. 2F. GlyPro-mTGase_Y75N-SL; Reaction time=90 m; Selectivity=15.66; hGH conversion rate=50%.

FIG. 2G. GlyPro-mTGase_Y62H_Y75N-SL; Reaction time=75 m; Selectivity=26.33; hGH conversion rate=38%.

FIG. 2H. GlyPro-mTGase_Y62H_Y75F-SL; Reaction time=120 m; Selectivity=36.21; hGH conversion rate=49.4%

FIG. 3. Analysis of reaction mixture of hGH mutants catalyzed by S. ladakanum TGase by HPLC. Top: hGH-Q40N. The first peak (26.5 min, area 1238) is product-Q141 and the second peak (29.7 min, area 375) is the remaining hGH-Q40N. Bottom: hGH-Q141N. The first peak (19.2 min, area 127) is product-Q40 and the second peak (30.3 min, area 1158) is the remaining hGH-Q141N.

FIG. 4. Analysis of reaction mixture of hGH mutants catalyzed by S. mobarense TGase by HPLC. Top: hGH-Q40N. The first peak (26.9 min, area 1283) is product-Q141 and the second peak (30.1 min, area 519) is the remaining hGH-Q40N. Bottom: hGH-Q141N. The first peak (19.5 min, area 296) is product-Q40 and the second peak (30.6 min, area 1291) is the remaining hGH-Q141N.

FIG. 5: CIE HPLC of transamination mixtures 3 and 4 from Table 5. Peak 1=hGH, peak 2=Transaminated in position 40, peak 3=Transaminated in position 141 and peak 4=Transaminated in positions 40/141.

### DESCRIPTION OF THE INVENTION

The present invention relates to peptides with TGase activity, which peptides have an improved selectivity for Gln141 in hGH over Gln40 in hGH, more specifically, the present invention relates to a transglutaminase peptide having a specificity for Gln-141 of hGH compared to Gln-40 of hGH, which is higher than the specificity of a peptide having an amino acid sequence as shown in SEQ ID No. 1 for Gln-141 of hGH compared to Gln-40 of hGH.

The terms “polypeptide” and “peptide” are used interchangeably herein and should be taken to mean a compound composed of at least five constituent amino acids connected by peptide bonds. The constituent amino acids may be from the group of the amino acids encoded by the genetic code and they may be natural amino acids which are not encoded by the genetic code, as well as synthetic amino acids. Natural amino acids which are not encoded by the genetic code are e.g. hydroxyproline, y-carboxyglutamate, ornithine, phosphoserine, D-alanine and D-glutamine. Synthetic amino acids comprise amino acids manufactured by chemical synthesis, i.e. D-isomers of the amino acids encoded by the genetic code such as D-alanine and D-leucine, Aib (a-aminoisobutyric acid), Abu (a-aminobutyric acid), Tle (tert-butylglycine), β-alanine, 3-aminomethyl benzoic acid and anthranilic acid. The term “conjugate” as a noun is intended to indicate a modified peptide, i.e. a peptide with a moiety bonded to it to modify the properties of said peptide. As verbs, the terms are intended to indicate the process of bonding a moiety to a peptide to modify the properties of said peptide.

In the present context a “peptide with TGase activity” or “transglutaminase” or similar is intended to mean a peptide having the ability to catalyze the acyl transfer reaction between the γ-carboxyamide group of glutamine residues and various primary amines, which acts as amine donors.

In the present context “transamination”, “transglutamination”, “transglutaminase reaction” or similar is intended to indicate a reaction where γ-glutaminyl of a glutamine residue from a protein/peptide is transferred to a primary amine or the E-amino group of lysine or water where an ammonia molecule is released.

In the present context, the terms “specificity” and “selectivity” are used interchangeably to describe a preference of the TGase for reacting with one or more specific glutamine residues in hGH as compared to other specific glutamine residues in hGH. For the purpose of this specification, the specificity of the peptides of the invention for Gln-40 as compared to Gln141 in hGH is decided according to the results of testing the peptides as described in the Examples.

The peptides of the present invention are useful as transglutaminases for transglutaminating peptides, for instance hGH. Transglutaminations of peptides are for instance useful for preparing conjugates of said peptides as described in WO2005/070468 and WO2006/134148.

One way of preparing conjugated peptides using hGH as an example comprises a first reaction between hGH and an amine donor comprising a functional group to afford a functionalised hGH, said first reaction being mediated (i.e. catalysed) by a TGase. In a second reaction step, said functionalised hGH is further reacted with e.g. a PEG or fatty acid capable or reacting with said incorporated functional group to provide conjugated hGH. The first reaction is sketched below.

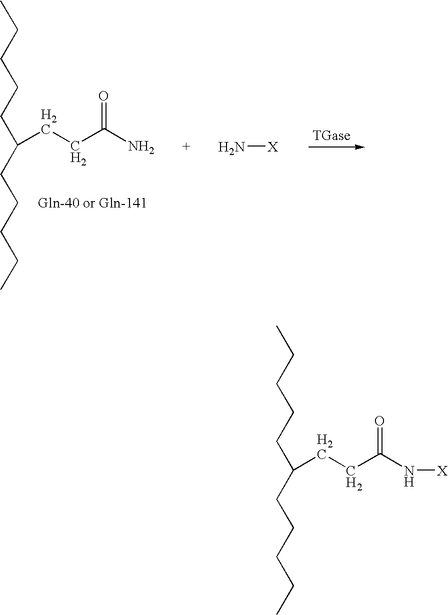

X represents a functional group or a latent functional group, i.e. a group which upon further reaction, e.g. oxidation or hydrolysation is transformed into a functional group.

The micro-organism S. mobaraensis is also classified as Streptoverticillium mobaraense. A TGase may be isolated from the organism, and this TGase is characterised by a relatively low molecular weight (˜38 kDa) and by being calcium-independent. The TGase from S. mobaraensis is relatively well-described; for instance has the crystal structure been solved (U.S. Pat. No. 156,956; Appl. Microbiol. Biotech. 64, 447-454 (2004)).

When the reaction above is mediated by TGase from Streptomyces mobaraensis, the reaction between hGH and H₂N—X (the amine donor) takes place predominately at Gln-40 and Gln-141. The above reaction may be employed to e.g. PEGylate hGH to achieve a therapeutic growth hormone product with a prolonged half life. As it is generally held desirable that therapeutic compositions are single-compound compositions, the above discussed lack of specificity requires a further purification step wherein Gln-40 functionalised hGH, Gln-141 functionalised hGH and/or Gln-40/Gln-141 double-functionalised hGH are separated from each other.

Such use of transglutaminases for conjugations of human growth hormone is extensively described in WO2005/070468, WO2006/134148, WO2006EP065440 and WO2006EP065439.

The sequence of a TGase isolated from S. ladakanum has an amino acid sequence which is identical to the sequence from S. mobaraensis except for a total of 22 amino acid differences between the two sequences (Yi-Sin Lin et al., Process Biochemistry 39(5), 591-598 (2004).

The sequence of the mTGase from S. ladakanum is given in SEQ ID No. 1 and the sequence of the mTGase from S. mobaraensis is given in SEQ ID No. 2.

The peptides of the present invention have a specificity for Gln-141 compared to Gln-40 of hGH, which is significantly higher than the specificity for Gln-141 compared to Gln-40 of hGH of a peptide having an amino acid sequence as shown in SEQ ID No. 2, wherein the specificity is measured as described in the Examples. Peptides of the present invention may thus be used in a method for transglutaminating hGH to increase production of Gln-40 functionalised hGH or Gln-141 functionalised hGH as compared to a reaction using a TGase having the amino acid sequence of SEQ ID No.2.

Thus, in one embodiment, a transglutaminase peptide of the invention has a specificity for Gln-141 of hGH compared to Gln-40 of hGH, which is higher than the specificity for Gln-141 of hGH compared to Gln-40 of hGH of a peptide having an amino acid sequence as shown in SEQ ID No. 2. In one embodiment, the specificity for a peptide of the present invention for Gln-141 compared to Gln-40 is at least 1.25, such as at least 1.50, for instance at least 1.75, such as at least 2.0, for instance at least 2.5, such as at least 3.0, for instance at least 3.5, such as at least 4.0, for instance at least 4.5, such as at least 5.0, for instance at least 5.5, such as at least 6.0, for instance at least 6.5, such as at least 7.0, for instance at least 7.5, such as at least 8.0, for instance at least 8.5, such as at least 9.0, for instance at least 9.5, such as at least 10.0 times higher than the specificity of a peptide having an amino acid sequence as shown in SEQ ID No. 2 for Gln-141 compared to Gln-40.

In one embodiment, a transglutaminase peptide of the invention has a specificity for Gln-141 of hGH compared to Gln-40 of hGH, which is higher than the specificity for Gln-141 of hGH compared to Gln-40 of hGH of a peptide having an amino acid sequence as shown in SEQ ID No. 1, or a peptide having the amino acid sequence as shown in SEQ ID No. 1 with the N-terminal addition of Ala-Pro, as a peptide having the amino acid sequence as shown in SEQ ID No. 1 with the N-terminal addition of Ala-Pro has the same specificity as a peptide having an amino acid sequence as shown in SEQ ID No. 1 (see Examples). In one embodiment, the specificity for a peptide of the present invention for Gln-141 compared to Gln-40 is at least 1.25, such as at least 1.50, for instance at least 1.75, such as at least 2.0, for instance at least 2.5, such as at least 3.0, for instance at least 3.5, such as at least 4.0, for instance at least 4.5, such as at least 5.0, for instance at least 5.5, such as at least 6.0, for instance at least 6.5, such as at least 7.0, for instance at least 7.5, such as at least 8.0, for instance at least 8.5, such as at least 9.0, for instance at least 9.5, such as at least 10.0 times higher than the specificity of a peptide having an amino acid sequence as shown in SEQ ID No. 1 for Gln-141 compared to Gln-40.

In one embodiment, a peptide according to the present invention comprises a sequence based on the sequence of the mTGase from S. ladakanum carrying mutations in specific amino acid residues and/or having additional N-terminally added amino acid residues. In one embodiment, a peptide according to the present invention comprises a sequence based on the sequence of the mTGase from S. mobaraensis additional with N-terminally added amino acid residues.

The present invention particularly relates to novel variants of transglutaminase from Streptoverticillium ladakanum. The variants may be used for site-specific modification of peptides at designated glutamine residues with improved selectivity.

In the present context, the term “variant” is intended to refer to either a naturally occurring variation of a given polypeptide or a recombinantly prepared or otherwise modified variation of a given peptide or protein in which one or more amino acid residues have been modified by amino acid substitution, addition, deletion, insertion or invertion.

In one embodiment, the invention provides an isolated peptide comprising an amino acid sequence having at least 80%, such as at least 85%, for instance at least 90%, such as at least 95%, for instance 100% identity with the amino acid sequence in SEQ ID No. 1, wherein said sequence is modified in one or more of the positions to the amino acid residues Tyr62, Tyr75 and Ser250 of SEQ ID No. 1.

The term “identity” as known in the art, refers to a relationship between the sequences of two or more peptides, as determined by comparing the sequences. In the art, “identity” also means the degree of sequence relatedness between peptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Preferred methods to determine identity are designed to give the largest match between the sequences tested. Methods to determine identity are described in publicly available computer programs. Preferred computer program methods to determine identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

For example, using the computer algorithm GAP (Genetics Computer Group, University of Wisconsin, Madison, Wis.), two peptides for which the percent sequence identity is to be determined are aligned for optimal matching of their respective amino acids (the “matched span”, as determined by the algorithm). A gap opening penalty (which is calculated as 3.times. the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp. 3 (1978) for the PAM 250 comparison matrix; Henikoff et al., Proc. Natl. Acad. Sci USA 89, 10915-10919 (1992) for the BLOSUM 62 comparison matrix) is also used by the algorithm.

Preferred parameters for a peptide sequence comparison include the following:

Algorithm: Needleman et al., J. Mol. Biol. 48, 443-453 (1970); Comparison matrix: BLOSUM 62 from Henikoff et al., PNAS USA 89, 10915-10919 (1992); Gap Penalty: 12, Gap Length Penalty: 4, Threshold of Similarity: 0.

The GAP program is useful with the above parameters. The aforementioned parameters are the default parameters for peptide comparisons (along with no penalty for end gaps) using the GAP algorithm.

In one embodiment, the invention provides an isolated peptide as described above, wherein said amino acid sequence is modified in the position corresponding to Tyr62, wherein the modification consists of a substitution of the original tyrosine residue with an amino acid residue different from Tyr. In one embodiment, the modification of the amino acid residue in the position corresponding to Tyr62 consists of a substitution of the original tyrosine residue with an amino acid residue selected from Ala, Arg, Asn, Asp, Cys, Gln, Glu, Gly, His, Ile, Leu, Lys, Met, Phe, Pro, Ser, Thr, Trp, and Val. In one embodiment, the Tyr in the position corresponding to Tyr62 is substituted with an amino acid residue selected from His, Met, Asn, Val, Thr, and Leu.

In one embodiment, the invention provides an isolated peptide as described above, wherein said amino acid sequence is modified in the position corresponding to Tyr75, wherein the modification consists of a substitution of the original tyrosine residue with an amino acid residue different from Tyr. In one embodiment, the modification of the amino acid residue in the position corresponding to Tyr75 consists of a substitution of the original tyrosine residue with an amino acid residue selected from Ala, Arg, Asn, Asp, Cys, Gln, Glu, Gly, His, Ile, Leu, Lys, Met, Phe, Pro, Ser, Thr, Trp, and Val. In one embodiment, the Tyr in the position corresponding to Tyr75 is substituted with Ala, Phe, Asn, Met, or Cys.

In one embodiment, the invention provides an isolated peptide as described above, wherein said amino acid sequence is modified in the position corresponding to Ser250, wherein the modification consists of a substitution of the original serine residue with an amino acid residue different from Ser. In one embodiment, the modification of the amino acid residue in the position corresponding to Ser250 consists of a substitution of the original tyrosine residue with an amino acid residue selected from Ala, Arg, Asn, Asp, Cys, Gln, Glu, Gly, His, Ile, Leu, Lys, Met, Phe, Pro, Thr, Trp, Tyr, and Val.

In one embodiment, the invention relates to an isolated peptide comprising an amino acid sequence having at least 80%, such as at least 85%, for instance at least 90%, such as at least 95%, for instance 100% identity with the amino acid sequence in SEQ ID No. 1, wherein said sequence is modified by the addition of one or more, such as from one to nine, for instance from one to eight, such as from one to seven, for instance from one to six, such as from one to five, for instance from one to four, such as from one to three, for instance from one to two, such as one amino acid in the N-terminal. In one embodiment, said sequence is modified by the addition of a Met in the N-terminal.

In one embodiment, the invention relates to an isolated peptide comprising an amino acid sequence having at least 80%, such as at least 85%, for instance at least 90%, such as at least 95%, for instance 100% identity with the amino acid sequence in SEQ ID No. 1, wherein said sequence is modified by the addition of one or more, such as from two to nine, for instance from two to eight, such as from two to seven, for instance from two to six, such as from two to five, for instance from two to four, such as from two to three, for instance two amino acids in the N-terminal. In one embodiment, the added amino acid residues is the dipeptide radical Gly-Pro-. In one embodiment, the added amino acid residues is the dipeptide radical Ala-Pro-.

In one embodiment, the invention relates to an isolated peptide comprising an amino acid sequence having at least 80%, such as at least 85%, for instance at least 90%, such as at least 95%, for instance 100% identity with the amino acid sequence in SEQ ID No. 2, wherein said sequence is modified by the addition of one or more, such as from one to nine, for instance from one to eight, such as from one to seven, for instance from one to six, such as from one to five, for instance from one to four, such as from one to three, for instance from one to two, such as one amino acid in the N-terminal. In one embodiment, said sequence is modified by the addition of a Met in the N-terminal.

In one embodiment, the invention relates to an isolated peptide comprising an amino acid sequence having at least 80%, such as at least 85%, for instance at least 90%, such as at least 95%, for instance 100% identity with the amino acid sequence in SEQ ID No. 2, wherein said sequence is modified by the addition of one or more, such as from two to nine, for instance from two to eight, such as from two to seven, for instance from two to six, such as from two to five, for instance from two to four, such as from two to three, for instance two amino acids in the N-terminal. In one embodiment, the added amino acid residues is the dipeptide radical Gly-Pro-. In one embodiment, the added amino acid residues is the dipeptide radical Ala-Pro-.

In one embodiment, the amino acid sequence having at least 80%, such as at least 85%, for instance at least 90%, such as at least 95%, for instance 100% identity with the amino acid sequence in SEQ ID No. 2, is the amino acid sequence of any of the TGases disclosed in WO2007020290. The present invention thus provides such TGases, which have been modified by the addition of from one to ten amino acids in the N-terminal.

The present invention also provides a TGase having the amino acid sequence of SEQ ID No. 1.

The peptides of the present invention exhibit TGase activity as determined in the assay described in U.S. Pat. No. 5,156,956. Briefly described, the measurement of the activity of a given peptide is carried out by performing a reaction using benzyloxycarbonyl-L-glutaminyl glycine and hydroxylamine as substrates in the absence of Ca²⁺, forming an iron complex with the resulting hydroxamic acid in the presence of trichloroacetic acid, measuring absorption at 525 nm and determining the amount of hydroxamic acid by a calibration curve to calculate the activity. For the purpose of this specification, a peptide, which exhibits transglutaminase activity in said assay, is deemed to have transglutaminase activity. In particular, the peptides of the present invention exhibit an activity which is more than 30%, such as more than 50%, such as more than 70%, such as more than 90% of that of a TGase from S. ladakanum having an amino acid sequence of SEQ ID No. 2.

The peptides of the present invention may be prepared in different ways. The peptides may be prepared by protein synthetic methods known in the art. If the peptides are rather large, this may be done more conveniently by synthesising several fragments of the peptides which are then combined to provide the peptides of the present invention. In a particular embodiment, however, the peptides of the present invention are prepared by fermentation of a suitable host comprising a nucleic acid construct encoding the peptides of the present invention.

In one embodiment, the present invention provides a nucleic acid construct encoding a peptide according to the present invention.

As used herein the term “nucleic acid construct” is intended to indicate any nucleic acid molecule of cDNA, genomic DNA, synthetic DNA or RNA origin. The term “construct” is intended to indicate a nucleic acid segment which may be single- or double-stranded, and which may be based on a complete or partial naturally occurring nucleotide sequence encoding a protein of interest. The construct may optionally contain other nucleic acid segments.

The nucleic acid construct of the invention encoding the peptide of the invention may suitably be of genomic or cDNA origin, for instance obtained by preparing a genomic or cDNA library and screening for DNA sequences coding for all or part of the protein by hybridization using synthetic oligonucleotide probes in accordance with standard techniques (cf. J. Sambrook et al, 1989, Molecular Cloning, A Laboratory Manual, 2d edition, Cold Spring Harbor, N.Y.) and by introducing the mutations as it is known in the art.

The nucleic acid construct of the invention encoding the protein may also be prepared synthetically by established standard methods, e.g. the phosphoamidite method described by Beaucage and Caruthers, Tetrahedron Letters 22, 1859-1869 (1981), or the method described by Matthes et al., EMBO Journal 3, 801-805 (1984). According to the phosphoamidite method, oligonucleotides are synthesized, e.g. in an automatic DNA synthesizer, purified, annealed, ligated and cloned in suitable vectors.

Furthermore, the nucleic acid construct may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire nucleic acid construct, in accordance with standard techniques.

The nucleic acid construct may also be prepared by polymerase chain reaction using specific primers, for instance as described in U.S. Pat. No. 4,683,202 or Saiki et al., Science 239, 487-491 (1988).

The nucleic acid construct is preferably a DNA construct which term will be used exclusively in the following.

In one embodiment, the present invention provides a recombinant vector comprising a nucleic acid construct according to the present invention.

In one embodiment, the present invention provides a host comprising the vector according to the present invention.

The recombinant vector into which the DNA construct of the invention is inserted may be any vector which may conveniently be subjected to recombinant DNA procedures, and the choice of vector will often depend on the host cell into which it is to be introduced. Thus, the vector may be an autonomously replicating vector, i.e. a vector which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the protein of the invention is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the protein.

The promoter may be any DNA sequence which shows transcriptional activity in the host cell of choice and may be derived from genes encoding proteins either homologous or heterologous to the host cell.

Examples of suitable promoters for use in yeast host cells include promoters from yeast glycolytic genes (Hitzeman et al., J. Biol. Chem. 255, 12073-12080 (1980); Alber and Kawasaki, J. Mol. Appl. Gen. 1, 419-434 (1982)) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4c (Russell et al., Nature 304, 652-654 (1983)) promoters.

Examples of suitable promoters for use in filamentous fungus host cells are, for instance, the ADH3 promoter (McKnight et al., The EMBO J. 4, 2093-2099 (1985)) or the tpiA promoter. Examples of other useful promoters are those derived from the gene encoding A. oryzae TAKA amylase, Rhizomucor miehei aspartic proteinase, A. niger neutral α-amylase, A. niger acid stable α-amylase, A. niger or A. awamori glucoamylase (gluA), Rhizomucor miehei lipase, A. oryzae alkaline protease, A. oryzae triose phosphate isomerase or A. nidulans acetamidase. Preferred are the TAKA-amylase and gluA promoters.

Examples of suitable promoters for use in bacterial host cells include the promoter of the Bacillus stearothermophilus maltogenic amylase gene, the Bacillus licheniformis alpha-amylase gene, the Bacillus amyloliquefaciens BAN amylase gene, the Bacillus subtilis alkaline protease gene, or the Bacillus pumilus xylosidase gene, or by the phage Lambda P_{R} or P_{L} promoters or the E. coli lac, trp or tac promoters.

The DNA sequence encoding the protein of the invention may also, if necessary, be operably connected to a suitable terminator, such as the human growth hormone terminator (Palmiter et al., op. cit.) or (for fungal hosts) the TPI1 (Alber and Kawasaki, op. cit.) or ADH3 (McKnight et al., op. cit.) terminators. The vector may further comprise elements such as polyadenylation signals (e.g. from SV40 or the adenovirus 5 Elb region), transcriptional enhancer sequences (e.g. the SV40 enhancer) and translational enhancer sequences (e.g. the ones encoding adenovirus VA RNAs).

The recombinant vector of the invention may further comprise a DNA sequence enabling the vector to replicate in the host cell in question.

When the host cell is a yeast cell, suitable sequences enabling the vector to replicate are the yeast plasmid 2μ replication genes REP 1-3 and origin of replication.

When the host cell is a bacterial cell, sequences enabling the vector to replicate are DNA polymerase III complex encoding genes and origin of replication.

The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 125-130 (1985)), or one which confers resistance to a drug, e.g. ampicillin, kanamycin, tetracyclin, chloramphenicol, neomycin, hygromycin or methotrexate. For filamentous fungi, selectable markers include amdS, pyrG, argB, niaD and sC.

To direct a protein of the present invention into the secretory pathway of the host cells, a secretory signal sequence (also known as a leader sequence, prepro sequence or pre sequence) may be provided in the recombinant vector. The secretory signal sequence is joined to the DNA sequence encoding the protein in the correct reading frame. Secretory signal sequences are commonly positioned 5′ to the DNA sequence encoding the protein. The secretory signal sequence may be that normally associated with the protein or may be from a gene encoding another secreted protein.

For secretion from yeast cells, the secretory signal sequence may encode any signal peptide which ensures efficient direction of the expressed protein into the secretory pathway of the cell. The signal peptide may be a naturally occurring signal peptide, or a functional part thereof, or it may be a synthetic peptide. Suitable signal peptides have been found to be the α-factor signal peptide (cf. U.S. Pat. No. 4,870,008), the signal peptide of mouse salivary amylase (cf. O. Hagenbuchle et al., Nature 289, 643-646 (1981)), a modified carboxypeptidase signal peptide (cf. L. A. Valls et al., Cell 48, 887-897 (1987)), the yeast BAR1 signal peptide (cf. WO 87/02670), or the yeast aspartic protease 3 (YAP3) signal peptide (cf. M. Egel-Mitani et al., Yeast 6, 127-137 (1990)).

For efficient secretion in yeast, a sequence encoding a leader peptide may also be inserted downstream of the signal sequence and upstream of the DNA sequence encoding the protein. The function of the leader peptide is to allow the expressed protein to be directed from the endoplasmic reticulum to the Golgi apparatus and further to a secretory vesicle for secretion into the culture medium (i.e. exportation of the protein across the cell wall or at least through the cellular membrane into the periplasmic space of the yeast cell). The leader peptide may be the yeast α-factor leader (the use of which is described in e.g. U.S. Pat. No. 4,546,082, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase.

The procedures used to ligate the DNA sequences coding for the present protein, the promoter and optionally the terminator and/or secretory signal sequence, respectively, and to insert them into suitable vectors containing the information necessary for replication, are well known to persons skilled in the art (cf., for instance, Sambrook et al., op. cit.).

The host cell into which the DNA construct or the recombinant vector of the invention is introduced may be any cell which is capable of producing the present protein and includes bacteria, yeast, fungi and higher eukaryotic cells.

Examples of bacterial host cells which, on cultivation, are capable of producing the protein of the invention are gram positive bacteria such as strains of Bacillus, such as strains of B. subtilis, B. licheniformis, B. lentus, B. brevis, B. stearothermophilus, B. alkalophilus, B. amyloliquefaciens, B. coagulans, B. circulans, B. lautus, B. megatherium or B. thuringiensis, or strains of Streptomyces, such as S. lividans or S. murinus, or gram negative bacteria such as Escherichia coli. The transformation of the bacteria may be effected by protoplast transformation or by using competent cells in a manner known per se (cf. Sambrook et al., supra). Other suitable hosts include S. mobaraensis, S. lividans, and C. glutamicum (Appl. Microbiol. Biotechnol. 64, 447-454 (2004)).

When expressing the protein in bacteria such as E. coli, the protein may be retained in the cytoplasm, typically as insoluble granules (known as inclusion bodies), or may be directed to the periplasmic space by a bacterial secretion sequence. In the former case, the cells are lysed and the granules are recovered and denatured after which the protein is refolded by diluting the denaturing agent. In the latter case, the protein may be recovered from the periplasmic space by disrupting the cells, e.g. by sonication or osmotic shock, to release the contents of the periplasmic space and recovering the protein.

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous proteins there from are described, e.g. in U.S. Pat. No. 4,599,31 1, U.S. Pat. No. 4,931,373, U.S. Pat. Nos. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequence encoding the protein of the invention may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 3459-3465 (1986); U.S. Pat. No. 4,882,279).

Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277 and EP 230 023. The transformation of F. oxysporum may, for instance, be carried out as described by Malardier et al. Gene 78, 147-156 (1989).

When a filamentous fungus is used as the host cell, it may be transformed with the DNA construct of the invention, conveniently by integrating the DNA construct in the host chromosome to obtain a recombinant host cell. This integration is generally considered to be an advantage as the DNA sequence is more likely to be stably maintained in the cell. Integration of the DNA constructs into the host chromosome may be performed according to conventional methods, e.g. by homologous or heterologous recombination.

The transformed or transfected host cell described above is then cultured in a suitable nutrient medium under conditions permitting the expression of the present peptide, after which the resulting protein is recovered from the culture.

The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The protein produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. ion exchange chromatography, gelfiltration chromatography, affinity chromatography, or the like, dependent on the type of protein in question.

In one embodiment, the present invention provides a composition comprising a peptide according to the present invention.

In one embodiment, the present invention provides a method for conjugating a peptide, wherein said method comprises reacting said peptide with an amine donor in the presence of a peptide according to the present invention. In one embodiment, the peptide to be conjugated is a growth hormone. In one embodiment, the peptide is hGH or a variant or derivative thereof.

In the present context, the term “derivative” is intended to refer to a polypeptide or variant or fragment thereof which is modified, i. e., by covalent attachment of any type of molecule, preferably having bioactivity, to the parent polypeptide. Typical modifications are amides, carbohydrates, alkyl groups, acyl groups, esters, PEGylations and the like.

In one embodiment, the present invention provides a method for conjugating a growth hormone as described above, wherein the amount of growth hormone conjugated at the position corresponding to position Gln-141 of hGH as compared to the amount of hGH conjugated at the position corresponding to position Gln-40 of hGH is significantly increased in comparison with the amount of hGH conjugated at the position corresponding to position Gln-141 of hGH as compared to the amount of hGH conjugated at the position corresponding to position Gln-40, when a peptide having the amino acid sequence as shown in SEQ ID No.2 is used in said method instead of the peptide according to the present invention.

In one embodiment, the present invention provides a method for conjugating hGH, wherein the amount of growth hormone conjugated at the position corresponding to position Gln-141 of hGH as compared to the amount of hGH conjugated at the position corresponding to position Gln-40 of hGH is significantly increased in comparison with the amount of hGH conjugated at the position corresponding to position Gln-141 of hGH as compared to the amount of hGH conjugated at the position corresponding to position Gln-40, when a peptide having the amino acid sequence as shown in SEQ ID No.1 is used in said method instead of the peptide according to the present invention.

In one embodiment, the present invention provides a method for the preparation of a hGH conjugated at the position corresponding to position 141, wherein said method comprises reacting said hGH with an amine donor in the presence of a peptide according to the present invention.

In one embodiment of a method according to the present invention the conjugated hGH is used for the preparation of pegylated hGH, wherein said pegylation takes place at the conjugated position.

In one embodiment, the present invention provides a method for the pharmaceutical preparation of a conjugated growth hormone, which method comprises a step of reacting said hGH or variant or derivative thereof with an amine donor in the presence of a peptide according to the present invention. In one embodiment, the growth hormone is hGH or a variant or derivative thereof.

In one embodiment, the present invention provides a method for the pharmaceutical preparation of a pegylated growth hormone, which method comprises a step of reacting said hGH or variant or derivative thereof with an amine donor in the presence of a peptide according to the present invention, and using the resulting conjugated growth hormone peptide for the preparation of a pegylated growth hormone, wherein said pegylation takes place at the conjugated position. In one embodiment, the growth hormone is hGH or a variant or derivative thereof. In one embodiment, the pegylated growth hormone is hGH pegylated in position Gln141. In one embodiment, the pegylated growth hormone is a pegylated growth hormone as described in WO2006/134148.

In one embodiment, the present invention provides the use of a peptide according to the present invention in the preparation of a conjugated growth hormone. In one embodiment, the growth hormone is hGH or a variant or derivative thereof. In one embodiment, the growth hormone is conjugated in the position corresponding to position Gln141 in hGH.

In one embodiment, the present invention provides a method for treatment of a disease or disorder related to lack of growth hormone in a patient, which method comprises administration of a pharmaceutical preparation as prepared by use of a method according to the present invention, wherein the peptide to be conjugated is a growth hormone, to a patient in need thereof. In one embodiment, the disease or disorder related to lack of growth hormone in a patient is selected from growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1st toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Crohn's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; or short stature due to glucocorticoid treatment in children.

The following is a list of embodiments of the present invention, which list is not to be construed as limiting:

Embodiment 1: A transglutaminase peptide having a specificity for Gln-141 of hGH compared to Gln-40 of hGH, which is higher than the specificity of a peptide having an amino acid sequence as shown in SEQ ID No. 1 for Gln-141 of hGH compared to Gln-40 of hGH.

Embodiment 2: A transglutaminase peptide according to embodiment 1 comprising an amino acid sequence having at least 80% identity with the amino acid sequence in SEQ ID No. 1.

Embodiment 3: A transglutaminase peptide according to embodiment 2 comprising an amino acid sequence having at least 85% identity with the amino acid sequence in SEQ ID No. 1.

Embodiment 4: A transglutaminase peptide according to embodiment 3 comprising an amino acid sequence having at least 90% identity with the amino acid sequence in SEQ ID No. 1.

Embodiment 5: A transglutaminase peptide according to embodiment 4 comprising an amino acid sequence having at least 95% identity with the amino acid sequence in SEQ ID No. 1.

Embodiment 6: A transglutaminase peptide according to any of embodiments 2 to 5, wherein said amino acid sequence is modified in one or more of the positions corresponding to the amino acid residues Tyr62, Tyr75 and Ser250 of SEQ ID No. 1.

Embodiment 7: An isolated peptide comprising an amino acid sequence having at least 80% identity with the amino acid sequence in SEQ ID No. 1, wherein said sequence is modified in one or more of the positions to the amino acid residues Tyr62, Tyr75 and Ser250 of SEQ ID No. 1.

Embodiment 8: An isolated peptide according to embodiment 7 comprising an amino acid sequence having at least 85% identity with the amino acid sequence in SEQ ID No. 1, wherein said sequence is modified in one or more of the positions corresponding to the amino acid residues Tyr62, Tyr75 and Ser250 of SEQ ID No. 1.

Embodiment 9: An isolated peptide according to embodiment 8 comprising an amino acid sequence having at least 90% identity with the amino acid sequence in SEQ ID No. 1, wherein said sequence is modified in one or more of the positions corresponding to the amino acid residues Tyr62, Tyr75 and Ser250 of SEQ ID No. 1.

Embodiment 10: An isolated peptide according to embodiment 9 comprising an amino acid sequence having at least 95% identity with the amino acid sequence in SEQ ID No. 1, wherein said sequence is modified in one or more of the positions corresponding to the amino acid residues Tyr62, Tyr75 and Ser250 of SEQ ID No. 1.

Embodiment 11: An isolated peptide according to embodiment 10 comprising an amino acid sequence as defined in SEQ ID No. 1, wherein said sequence is modified in one or more of the positions corresponding to the amino acid residues Tyr62, Tyr75 and Ser250 of SEQ ID No. 1.

Embodiment 12: An isolated peptide according to any of embodiments 6 to 11, wherein said amino acid sequence is modified in the position corresponding to Tyr62, wherein the modification consists of a substitution of the original tyrosine residue with an amino acid residue different from Tyr.

Embodiment 13: An isolated peptide according to embodiment 12, wherein the modification of the amino acid residue in the position corresponding to Tyr62 consists of a substitution of the original tyrosine residue with an amino acid residue selected from Ala, Arg, Asn, Asp, Cys, Gln, Glu, Gly, His, Ile, Leu, Lys, Met, Phe, Pro, Ser, Thr, Trp, and Val.

Embodiment 14: An isolated peptide according to embodiment 13, wherein the Tyr in the position corresponding to Tyr62 is substituted with an amino acid residue selected from His, Met, Asn, Val, Thr, and Leu.

Embodiment 15: An isolated peptide according to embodiment 14, wherein the Tyr in the position corresponding to Tyr62 is substituted with His.

Embodiment 16: An isolated peptide according to embodiment 14, wherein the Tyr in the position corresponding to Tyr62 is substituted with Val.

Embodiment 17: An isolated peptide according to embodiment 14, wherein the Tyr in the position corresponding to Tyr62 is substituted with an amino acid residue selected from Met, Asn, Thr, and Leu.

Embodiment 18: An isolated peptide according to embodiment 17, wherein the Tyr in the position corresponding to Tyr62 is substituted with Met.

Embodiment 19: An isolated peptide according to embodiment 17, wherein the Tyr in the position corresponding to Tyr62 is substituted with Asn.

Embodiment 20: An isolated peptide according to embodiment 17, wherein the Tyr in the position corresponding to Tyr62 is substituted with Thr.

Embodiment 21: An isolated peptide according to embodiment 17, wherein the Tyr in the position corresponding to Tyr62 is substituted with Leu.

Embodiment 22: An isolated peptide according to any of embodiments 6 to 21, wherein said amino acid sequence is modified in the position corresponding to Tyr75, wherein the modification consists of a substitution of the original tyrosine residue with an amino acid residue different from Tyr.

Embodiment 23: An isolated peptide according to embodiment 22, wherein the modification of the amino acid residue in the position corresponding to Tyr75 consists of a substitution of the original tyrosine residue with an amino acid residue selected from Ala, Arg, Asn, Asp, Cys, Gln, Glu, Gly, His, Ile, Leu, Lys, Met, Phe, Pro, Ser, Thr, Trp, and Val.

Embodiment 24: An isolated peptide according to embodiment 23, wherein the Tyr in the position corresponding to Tyr75 is substituted with Ala, Phe, Asn, Met, Leu, or Cys.

Embodiment 25: An isolated peptide according to embodiment 24, wherein the Tyr in the position corresponding to Tyr75 is substituted with a Phe.

Embodiment 26: An isolated peptide according to embodiment 24, wherein the Tyr in the position corresponding to Tyr75 is substituted with an Asn.

Embodiment 27: An isolated peptide according to embodiment 24, wherein the Tyr in the position corresponding to Tyr75 is substituted with Ala, Met, Leu, or Cys.

Embodiment 28: An isolated peptide according to embodiment 27, wherein the Tyr in the position corresponding to Tyr75 is substituted with an Ala.

Embodiment 29: An isolated peptide according to embodiment 27, wherein the Tyr in the position corresponding to Tyr75 is substituted with a Met.

Embodiment 30: An isolated peptide according to embodiment 27, wherein the Tyr in the position corresponding to Tyr75 is substituted with a Leu.

Embodiment 31: An isolated peptide according to embodiment 27, wherein the Tyr in the position corresponding to Tyr75 is substituted with a Cys.

Embodiment 32: An isolated peptide according to any of embodiments 6 to 31, wherein said amino acid sequence is modified in the position corresponding to Ser250, wherein the modification consists of a substitution of the original serine residue with an amino acid residue different from Ser.

Embodiment 33: An isolated peptide according to embodiment 32, wherein the modification of the amino acid residue in the position corresponding to Ser250 consists of a substitution of the original serine residue with an amino acid residue selected from Ala, Arg, Asn, Asp, Cys, Gln, Glu, Gly, His, Ile, Leu, Lys, Met, Phe, Pro, Thr, Trp, Tyr, and Val.

Embodiment 34: An isolated peptide according to embodiment 33, wherein the modification of the amino acid residue in the position corresponding to Ser250 consists of a substitution of the original serine residue with an amino acid residue selected from Ala, Arg, Asp, Cys, Gln, Gly, His, Leu, Met, Phe, Pro, Thr, Trp, Tyr, and Val.

Embodiment 35: An isolated peptide according to embodiment 33, wherein the modification of the amino acid residue in the position corresponding to Ser250 consists of a substitution of the original serine residue with a Gly.

Embodiment 36: An isolated peptide according to embodiment 33 or embodiment 35, wherein the modification of the amino acid residue in the position corresponding to Ser250 consists of a substitution of the original serine residue with an amino acid residue selected from Cys, Leu, Pro, Trp, Tyr, and Val.

Embodiment 37: An isolated peptide according to embodiment 36, wherein said Ser250 is substituted with a Cys.

Embodiment 38: An isolated peptide according to embodiment 36, wherein said Ser250 is substituted with a Leu.

Embodiment 39: An isolated peptide according to embodiment 36, wherein said Ser250 is substituted with a Pro.

Embodiment 40: An isolated peptide according to embodiment 36, wherein said Ser250 is substituted with a Trp.

Embodiment 41: An isolated peptide according to embodiment 36, wherein said Ser250 is substituted with a Tyr.

Embodiment 42: An isolated peptide according to embodiment 36, wherein said Ser250 is substituted with a Val.

Embodiment 43: An isolated peptide comprising an amino acid sequence having at least 80% identity with the amino acid sequence in SEQ ID No. 1, wherein said sequence is modified by the addition of from one to ten amino acid residues in the N-terminal.

Embodiment 44: An isolated peptide according to embodiment 43 comprising an amino acid sequence having at least 85% identity with the amino acid sequence in SEQ ID No. 1, wherein said sequence is modified by the addition of from one to ten amino acids in the N-terminal.

Embodiment 45: An isolated peptide according to embodiment 44 comprising an amino acid sequence having at least 90% identity with the amino acid sequence in SEQ ID No. 1, wherein said sequence is modified by the addition of from one to ten amino acids in the N-terminal.

Embodiment 46: An isolated peptide according to embodiment 45 comprising an amino acid sequence having at least 95% identity with the amino acid sequence in SEQ ID No. 1, wherein said sequence is modified by the addition of from one to ten amino acids in the N-terminal.

Embodiment 47: An isolated peptide according to embodiment 46 comprising an amino acid sequence as defined in SEQ ID No. 1, wherein said sequence is modified by the addition of from one to ten amino acids in the N-terminal.

Embodiment 48: An isolated peptide according to any of embodiments 2 to 42, wherein said amino acid sequence is modified by the addition of from one to ten amino acids in the N-terminal.

Embodiment 49: An isolated peptide comprising an amino acid sequence having at least 80% identity with the amino acid sequence in SEQ ID No. 2, wherein said sequence is modified by the addition of from one to ten amino acids in the N-terminal.

Embodiment 50: An isolated peptide according to embodiment 49 comprising an amino acid sequence having at least 85% identity with the amino acid sequence in SEQ ID No. 2, wherein said sequence is modified by the addition of from one to ten amino acids in the N-terminal.

Embodiment 51: An isolated peptide according to embodiment 50 comprising an amino acid sequence having at least 90% identity with the amino acid sequence in SEQ ID No. 2, wherein said sequence is modified by the addition of from one to ten amino acids in the N-terminal.

Embodiment 52: An isolated peptide according to embodiment 51 comprising an amino acid sequence having at least 95% identity with the amino acid sequence in SEQ ID No. 2, wherein said sequence is modified by the addition of from one to ten amino acids in the N-terminal.

Embodiment 53: An isolated peptide according to embodiment 52 comprising an amino acid sequence as defined in SEQ ID No. 2, wherein said sequence is modified by the addition of from one to ten amino acids in the N-terminal.

Embodiment 54: An isolated peptide according to any of embodiments 49 to 53, wherein said sequence is further modified in one or more of the amino acid residues selected from the amino acid residues corresponding to positions Asp-4, Val-30, Tyr-62, Tyr-75, Arg-89, Glu-115, Ser-210, Asp-221, Ala-226, Pro-227, Gly-250, Val-252, Asn-253, Phe-254, His-277, Tyr-278, Leu-285, Tyr-302, Asp-304, and Lys-327 in SEQ ID No. 2.

Embodiment 55: An isolated peptide according to embodiment 54, wherein said sequence is modified in the amino acid residue corresponding to Gly-250 in SEQ ID No. 2.

Embodiment 56: An isolated peptide according to embodiment 55, wherein said Gly-250 is substituted with a Thr.

Embodiment 57: An isolated peptide according to embodiment 55, wherein said Gly-250 is substituted with a Ser.

Embodiment 58: An isolated peptide according to any of embodiments 49 to 57, wherein said sequence is further modified in one or more of the amino acid residues situated less than 20 Å away from the amino acid residue corresponding to Cys-64 in SEQ ID No. 2.

Embodiment 59: An isolated peptide according to embodiment 58, wherein said sequence is modified in one or more of the amino acid residues situated less than 15 Å away from the amino acid residue corresponding to Cys-64 in SEQ ID No. 2.

Embodiment 60: An isolated peptide according to embodiment 59, wherein the sequence is not modified in the position corresponding to position Cys64 in SEQ ID No. 2.

Embodiment 61: An isolated peptide according to embodiment 59 or 60, wherein said sequence is modified in one or more of the amino acid residues selected from the amino acid residues corresponding to positions Val-30, Tyr-62, Val-252, Asn-253, Phe-254, His-277, Tyr-278, and Leu-285 in SEQ ID No. 2.

Embodiment 62: An isolated peptide according to embodiment 61, wherein said sequence is modified in the amino acid residue corresponding to Tyr-62 in SEQ ID No. 2.

Embodiment 63: An isolated peptide according to embodiment 61 or embodiment 62, wherein said Tyr-62 is substituted with a His, Glu, le, Leu, Met, Asn, Gln, Thr, Val or Trp.

Embodiment 64: An isolated peptide according to any of embodiments 61 to 63, wherein said Tyr-62 is substituted with a Glu.

Embodiment 65: An isolated peptide according to any of embodiments 61 to 63, wherein said Tyr-62 is substituted with a Trp.

Embodiment 66: An isolated peptide according to any of embodiments 61 to 63, wherein said Tyr-62 is substituted with a His, Ile, Leu, Met, Asn, Gln, Thr, or Val.

Embodiment 67: An isolated peptide according to embodiment 66, wherein said Tyr-62 is substituted with a His.

Embodiment 68: An isolated peptide according to embodiment 66, wherein said Tyr-62 is substituted with a Glu.

Embodiment 69: An isolated peptide according to embodiment 66, wherein said Tyr-62 is substituted with a Ile.

Embodiment 70: An isolated peptide according to embodiment 66, wherein said Tyr-62 is substituted with a Met.

Embodiment 71: An isolated peptide according to embodiment 66, wherein said Tyr-62 is substituted with a Asn.

Embodiment 72: An isolated peptide according to embodiment 66, wherein said Tyr-62 is substituted with a Gln.

Embodiment 73: An isolated peptide according to embodiment 66, wherein said Tyr-62 is substituted with a Thr.

Embodiment 74: An isolated peptide according to embodiment 66, wherein said Tyr-62 is substituted with a Val.

Embodiment 75: An isolated peptide according to embodiment 66, wherein said Tyr-62 is substituted with a Trp.

Embodiment 76: An isolated peptide according to any of embodiments 61 to 75, wherein said sequence is modified in one or more of the amino acid residues corresponding to His-277 and Tyr-278 in SEQ ID No. 2.

Embodiment 77: An isolated peptide according to any of embodiments 61 to 76, wherein said sequence is modified in the amino acid residue corresponding to Leu-285 in SEQ ID No. 2.

Embodiment 78: An isolated peptide according to embodiment 77, wherein said Leu-285 is substituted with a Thr.

Embodiment 79: An isolated peptide according to any of embodiments 61 to 78, wherein said sequence is modified in one or more of the amino acid residues selected from the amino acid residues corresponding to positions Val-252, Asn-253, and Phe-254 in SEQ ID No. 2.

Embodiment 80: An isolated peptide according to any of embodiments 59 to 79, wherein said sequence is modified in the amino acid residue corresponding to Val-30 in SEQ ID No. 2.

Embodiment 81: An isolated peptide according to embodiment 80, wherein said Val-30 is substituted with an Ile in SEQ ID No. 2.

Embodiment 82: An isolated peptide according to embodiment 54, wherein said sequence is further modified in one or more of the amino acid residues selected from the amino acid residues corresponding to positions Asp-4, Arg-89, Glu-115, Ser-210, Asp-221, and Lys-327 in SEQ ID No. 2.

Embodiment 83: An isolated peptide according to embodiment 82, wherein said Asp-4 is substituted with an Glu.

Embodiment 84: An isolated peptide according to embodiment 82 or embodiment 83, wherein said Asp-4 is substituted with an Glu and the amino acids in positions 1, 2 and 3 have been deleted.

Embodiment 85: An isolated peptide according to any of embodiments 82 to 84, wherein the amino acid residue corresponding to Arg-89 in SEQ ID No. 2 is substituted with a Lys.

Embodiment 86: An isolated peptide according to any of embodiments 82 to 85, wherein the amino acid residue corresponding to Glu-115 in SEQ ID No. 2 is substituted with an Asp.

Embodiment 87: An isolated peptide according to any of embodiments 82 to 86, wherein the amino acid residue corresponding to Ser-210 in SEQ ID No. 2 is substituted with a Gly.

Embodiment 88: An isolated peptide according to any of embodiments 82 to 87, wherein the amino acid residue corresponding to Asp-221 in SEQ ID No. 2 is substituted with a Ser.

Embodiment 89: An isolated peptide according to any of embodiments 82 to 88, wherein the amino acid residue corresponding to Lys-327 in SEQ ID No. 2 is substituted with a Thr.

Embodiment 90: An isolated peptide according to any of embodiments 54 to 89, wherein said sequence is modified in one or more of the amino acid residues selected from the amino acid residues corresponding to positions Ala-226 and Pro-227 in SEQ ID No. 2.

Embodiment 91: An isolated peptide according to embodiment 90, wherein said Ala-226 is substituted with an Asp.

Embodiment 92: An isolated peptide according to embodiment 90, wherein the amino acid residue corresponding to Pro-227 in SEQ ID No. 2 is substituted with an Arg.

Embodiment 93: An isolated peptide according to any of embodiments 54 to 92, wherein said sequence is modified in the amino acid residue corresponding to Tyr-75 in SEQ ID No. 2.

Embodiment 94: An isolated peptide according to embodiment 93, wherein said Tyr-75 is substituted with an amino acid different from Glu.

Embodiment 95: An isolated peptide according to embodiment 94, wherein said Tyr-75 is substituted with an amino acid different from Asp or Glu.

Embodiment 96: An isolated peptide according to embodiment 95, wherein said Tyr-75 is substituted with an amino acid different from an acidic amino acid residue.

Embodiment 97: An isolated peptide according to any of embodiments 93 to 96, wherein said Tyr-75 is substituted with Ala.

Embodiment 98: An isolated peptide according to any of embodiments 93 to 96, wherein said Tyr-75 is substituted with Cys.

Embodiment 99: An isolated peptide according to any of embodiments 93 to 96, wherein said Tyr-75 is substituted with Phe.

Embodiment 100: An isolated peptide according to any of embodiments 93 to 96, wherein said Tyr-75 is substituted with Leu.

Embodiment 101: An isolated peptide according to any of embodiments 93 to 96, wherein said Tyr-75 is substituted with Met.

Embodiment 102: An isolated peptide according to any of embodiments 93 to 96, wherein said Tyr-75 is substituted with Asn.

Embodiment 103: An isolated peptide according to any of embodiments 93 to 96, wherein said Tyr-75 is substituted with Pro.

Embodiment 104: An isolated peptide according to any of embodiments 93 to 96, wherein said Tyr-75 is substituted with Ser.

Embodiment 105: An isolated peptide according to any of embodiments 54 to 104, wherein said sequence is modified in the amino acid residue corresponding to Tyr-302 in SEQ ID No. 2.

Embodiment 106: An isolated peptide according to embodiment 105, wherein said Tyr-302 is substituted with a basic amino acid residue different from Tyr.

Embodiment 107: An isolated peptide according to embodiment 106, wherein said Tyr-302 is substituted with Arg or Lys.

Embodiment 108: An isolated peptide according to embodiment 107, wherein said Tyr-302 is substituted with Arg.

Embodiment 109: An isolated peptide according to any of embodiments 54 to 108, wherein said sequence is modified in the amino acid residue corresponding to Asp-304 in SEQ ID No. 2.

Embodiment 110: An isolated peptide according to embodiment 109, wherein said Asp-304 is substituted with a basic amino acid residue.

Embodiment 111: An isolated peptide according to embodiment 110, wherein said Asp-304 is substituted with Tyr, Lys or Arg.

Embodiment 112: An isolated peptide according to embodiment 111, wherein said Asp-304 is substituted with Lys.

Embodiment 113: An isolated peptide according to any of embodiments 43 to 112, wherein said sequence is modified by the addition of from one to nine amino acids in the N-terminal.

Embodiment 114: An isolated peptide according to embodiment 113, wherein said sequence is modified by the addition of from one to eight amino acids in the N-terminal.

Embodiment 115: An isolated peptide according to embodiment 114, wherein said sequence is modified by the addition of from one to seven amino acids in the N-terminal.

Embodiment 116: An isolated peptide according to embodiment 115, wherein said sequence is modified by the addition of from one to six amino acids in the N-terminal.

Embodiment 117: An isolated peptide according to embodiment 116, wherein said sequence is modified by the addition of from one to five amino acids in the N-terminal.

Embodiment 118: An isolated peptide according to embodiment 117, wherein said sequence is modified by the addition of from one to four amino acids in the N-terminal.

Embodiment 119: An isolated peptide according to embodiment 118, wherein said sequence is modified by the addition of from one to three amino acids in the N-terminal.

Embodiment 120: An isolated peptide according to embodiment 119, wherein said sequence is modified by the addition of from one to two amino acids in the N-terminal.

Embodiment 121: An isolated peptide according to embodiment 120, wherein said sequence is modified by the addition of one amino acid in the N-terminal.

Embodiment 122: An isolated peptide according to embodiment 121, wherein said sequence is modified by the addition of a Met in the N-terminal.

Embodiment 123: An isolated peptide according to any of embodiments 43 to 112, wherein said sequence is modified by the addition of from two to nine amino acids in the N-terminal.

Embodiment 124: An isolated peptide according to embodiment 123, wherein said sequence is modified by the addition of from two to eight amino acids in the N-terminal.

Embodiment 125: An isolated peptide according to embodiment 124, wherein said sequence is modified by the addition of from two to seven amino acids in the N-terminal.

Embodiment 126: An isolated peptide according to embodiment 125, wherein said sequence is modified by the addition of from two to six amino acids in the N-terminal.

Embodiment 127: An isolated peptide according to embodiment 126, wherein said sequence is modified by the addition of from two to five amino acids in the N-terminal.

Embodiment 128: An isolated peptide according to embodiment 127, wherein said sequence is modified by the addition of from two to four amino acids in the N-terminal.

Embodiment 129: An isolated peptide according to embodiment 128, wherein said sequence is modified by the addition of from two to three amino acids in the N-terminal.

Embodiment 130: An isolated peptide according to embodiment 129, wherein said sequence is modified by the addition of two amino acids in the N-terminal.

Embodiment 131: An isolated peptide according to embodiment 130, wherein the added dipeptide radical is Gly-Pro-.

Embodiment 132: An isolated peptide according to embodiment 130, wherein the added dipeptide radical is Ala-Pro-.

Embodiment 133: An isolated peptide according to any of embodiments 7 to 132, which peptide has transglutaminase activity.

Embodiment 134: An isolated peptide according to any of embodiments 1 to 6 or embodiment 133, which peptide has a specificity for Gln-141 of hGH compared to Gln-40 of hGH, which is higher than the specificity of a peptide having an amino acid sequence as shown in SEQ ID No. 2 for Gln-141 of hGH compared to Gln-40 of hGH.

Embodiment 135: An isolated peptide according to any of embodiments 1 to 6 or embodiment 133, which peptide has a specificity for Gln-141 of hGH compared to Gln-40 of hGH, which is higher than the specificity of a peptide having an amino acid sequence as shown in SEQ ID No. 1 for Gln-141 of hGH compared to Gln-40 of hGH.

Embodiment 136: An isolated peptide comprising the sequence of SEQ ID No. 1.

Embodiment 137: An isolated peptide having the sequence of SEQ ID No. 1.

Embodiment 138: A nucleic acid construct encoding a peptide according to any of embodiments 1 to 137.

Embodiment 139: A vector comprising the nucleic acid construct of embodiment 138.

Embodiment 140: A host cell comprising the vector of embodiment 139.

Embodiment 141: A composition comprising a peptide according to any of embodiments 1 to 137.

Embodiment 142: A method for conjugating a peptide, wherein said method comprises reacting said peptide with an amine donor in the presence of a peptide according to any of embodiments 1 to 137.

Embodiment 143: A method for conjugating a peptide according to embodiment 142, wherein said peptide to be conjugated is a growth hormone.

Embodiment 144: A method according to embodiment 143, wherein said growth hormone is hGH or a variant or derivative thereof.

Embodiment 145: A method for conjugating a growth hormone according to embodiment 144, wherein the amount of growth hormone conjugated at the position corresponding to position Gln-141 of hGH as compared to the amount of hGH conjugated at the position corresponding to position Gln-40 of hGH is significantly increased in comparison with the amount of hGH conjugated at the position corresponding to position Gln-141 of hGH as compared to the amount of hGH conjugated at the position corresponding to position Gln-40, when a peptide having the amino acid sequence as shown in SEQ ID No.2 is used in said method instead of the peptide according to any of embodiments 1 to 137.

Embodiment 146: A method for conjugating hGH according to embodiment 142, wherein the amount of growth hormone conjugated at the position corresponding to position Gln-141 of hGH as compared to the amount of hGH conjugated at the position corresponding to position Gln-40 of hGH is significantly increased in comparison with the amount of hGH conjugated at the position corresponding to position Gln-141 of hGH as compared to the amount of hGH conjugated at the position corresponding to position Gln-40, when a peptide having the amino acid sequence as shown in SEQ ID No.1 is used in said method instead of the peptide according to any of embodiments 1 to 137.

Embodiment 147: A method for the preparation of a hGH conjugated at the position corresponding to position 141, wherein said method comprises reacting said hGH with an amine donor in the presence of a peptide according to any of embodiments 1 to 137.

Embodiment 148: A method according to any of embodiments 142 to 147, wherein the conjugated hGH is used for the preparation of pegylated hGH, wherein said pegylation takes place at the conjugated position.

Embodiment 149: A method for the pharmaceutical preparation of a conjugated growth hormone, which method comprises a step of reacting said hGH or variant or derivative thereof with an amine donor in the presence of a peptide according to any of embodiments 1 to 137.

Embodiment 150: A method according to embodiment 149, wherein said growth hormone is hGH or a variant or derivative thereof.

Embodiment 151: A method for the pharmaceutical preparation of a pegylated growth hormone, which method comprises a step of reacting said hGH or variant or derivative thereof with an amine donor in the presence of a peptide according to any of embodiments 1 to 137, and using the resulting conjugated growth hormone peptide for the preparation of a pegylated growth hormone, wherein said pegylation takes place at the conjugated position.

Embodiment 152: A method according to embodiment 151, wherein said growth hormone is hGH or a variant or derivative thereof.

Embodiment 153: A method according to embodiment 152, wherein the pegylated growth hormone is hGH pegylated in position Gln141.

Embodiment 154: Use of a peptide according to any of embodiments 1 to 137 in the preparation of a conjugated growth hormone.

Embodiment 155: Use according to embodiment 154, wherein the growth hormone is hGH or a variant or derivative thereof.

Embodiment 156: Use according to embodiment 154 or embodiment 155, wherein the growth hormone is conjugated in the position corresponding to position Gln141 in hGH.

Embodiment 157: A method for treatment of a disease or disorder related to lack of growth hormone in a patient, which method comprises administration of a pharmaceutical preparation as prepared by use of a method according to any of embodiments 149 to 153 to a patient in need thereof.

Embodiment 158: A method according to embodiment 157, wherein the disease or disorder related to lack of growth hormone in a patient is selected from growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1st toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Crohn's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; or short stature due to glucocorticoid treatment in children.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context. For example, the phrase “the compound” is to be understood as referring to various “compounds” of the invention or particular described aspect, unless otherwise indicated.

Unless otherwise indicated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or aspect of the invention using terms such as “comprising”, “having,” “including,” or “containing” with reference to an element or elements is intended to provide support for a similar aspect or aspect of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

### EXAMPLES

### Example 1

### Cloning of Propeptide-mTGase in GlyPro-TGase Form and Mutant Generation

TGase from Streptoverticillium ladakanum ATCC27441

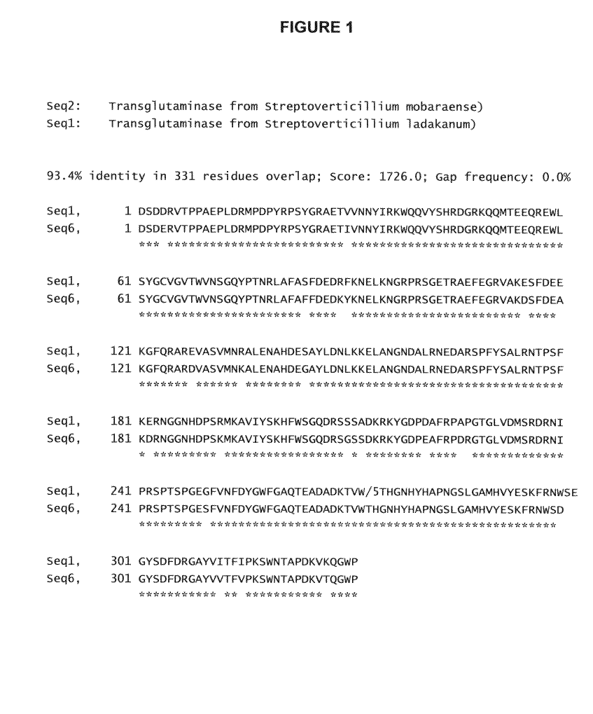

The sequence of Propeptide-mTGase from S. ladakanum (Propeptide-mTGase is the peptide, which is the result of the expression of the DNA encoding TGase from S. ladakanum in another organism, such as E. coli) is shown as SEQ ID No.3. The propeptide-part is aa 1-49 of SEQ ID No. 3 and the rest of sequence was the mature mTGase as shown in SEQ ID No. 1. The mature mTGase part (SEQ ID No. 1) has 93.4% identity to that of mTGase from S. mobaraensis (SEQ ID No. 2) as shown in FIG. 1.

A 3C-protease sequence LEVLFQGP (3C) was cloned between the propeptide-domain (aa 1-49 of SEQ ID No. 3) and mature mTGase domain of Propeptide-TGase of S. ladakanum. The 3C-protease cleaves specifically between the Q and the G of the LEVLFQGP site, which resulted in two additional amino acid residues, Gly-Pro to be added to the N-terminus of the mature mTGase (shown in SEQ ID No. 1). For expression in E. coli DNA encoding a Met-Propeptide-(3C)-mTGase was cloned between NdeI and BamHI sites of pET39b (Novagen) expression vector and transferred into E. coli BL21 (DE3) for expression.

Site-directed mutagenesis was performed using QuikChange site-directed mutagenesis kit (Stratagene). For example, the mutation of Y75A, Y75F, Y62H_Y75N and Y62H_Y75F (using the numbering of SEQ ID No. 1) were generated using DNA encoding Propeptide-(3C)-mTGase sequence as the template in PCR.

### Example 2

Selectivity of TGase Mutants with Added N-Terminally Amino Acid Residues

Preparation of GlyPro-mTGase

The pET39b_Met-Propeptide-(3C)-mTGase/E. coli BL21 (DE3) cells were cultivated at 30° C. in LB medium supplemented with 30 μg/ml kanamycin to an optical density of 0.4, and the cells were induced with 0.1 mM IPTG for another 4 h. The cell pellet was harvested by centrifugation.

The soluble fraction from the cell pellet was extracted and purified with anion exchange, Q-sepharose HP, column to obtain pure Propeptide-(3C)-mTGase protein. This protein was then digested with 3C-protease (from poliovirus) at 1:100 (w/w) ratio to the Propeptide-(3C)-mTGase protein at 20° C. for overnight. The digestion mixture was further purified by cation-exchange column, SP Sepharose HP/Source 30S, for active mTGase, which is identified by TGase activity assay.

Preparation of AlaPro-mTGase

AlaPro-mTGase was produced in a similar way as GlyPro-mTGase except the digestion of propeptide was achieved with enterokinase (EK) instead of 3C protease. Briefly, Propeptide-mTGase from Streptomyces mobaraensis was expressed in E. coli and was found in the soluble fraction. Propeptide-mTGase was purified by Q Sepharose HP ion exchange chromatography, and digested by EK to give AlaPro-mTGase. Then, AlaPro-mTGase was further purified on SP Sepharose HP ion exchange column.

To compare the effect of different N-terminal extra sequence on the selectivity of mTGase from S. ladakanum, mTGase in the forms of Met-mTGase, AlaPro-mTGase and wild type mTGase from S. ladakanum were cloned, expressed and purified separately.

Comparing to the AlaPro-mTGase from S. mobaraensis, which was generated from EK as described above, the generation of GlyPro-mTGase-SL was processed by 3C-protease (from poliovirus) digestion from Propeptide-3C-mTGase-SL, which is more specific with an improved recovery yield than using EK digestion.

### Example 3

Screening Assay for High Selective Variant—Kinetics Method Used to Evaluate the Effect of N-Terminal Extra Sequence to the Selectivity of mTGase from S. ladakanum

Preparation of hGHQ40N and hGHQ141N

hGH mutants hGHQ40N and hGHQ141N, were constructed by site-directed mutagenesis. They were expressed as MEAE-hGHQ40N and MEAE-hGHQ141N in E. coli with 4 additional amino acid residues at the N-terminus and purified in the same way as wild type recombinant hGH. In brief, the soluble MEAE-hGH mutants were recovered from crude E. coli lysates with Q Sepharose XL chromatography, then further polished with phenyl sepharose FF. The partial purified MEAE-hGH mutants were digested with DAP-1 enzyme at 42° for 1 hour to remove MEAE at N-terminus. Finally, the hGH mutants were precipitated with 38% cold ethanol, then dissolved with 7M urea, and purified with Source 30 Q column.

Kinetic Reaction

The kinetic reactions were carried out in 200 μl Tris-HCl buffer, 20 mM, pH 7.4 containing 200 mM NaCl, 50 uM hGHQ141N or hGHQ40N, 100 uM dansyl-cadaverine (DNC, Fluka). The reactions were started by adding 2 μg mTGase and run at 26° C. Fluorescence was monitored at Ex/Em: 340/520 nm every 20 sec for 1 hour. The progress curves were fitted with 2nd order polynomial using the data collected between 0-2000 s to obtain the slope. The fitting calculation is based on the data taken at earlier time ranges (0-2000 sec) where the slopes of progress curves are linear and the backward reaction is relatively minimal.

### Example 4

### Capillary Electrophoresis to Verify the High Selectivity of TGase Mutants:

Transglutamination Reaction of hGH

Transglutamination reaction was performed using 1,3-diamino-propanol as the amine donor. The reaction was started by the addition of TGase protein and incubated at room temperature for 2 h. Samples were taken at time intervals (15-30 m), frozen with liquid nitrogen and stored at −20° C. for the analysis of conversion rate and selectivity by CE. The reaction mixture was made as in Table 1.

TABLE 1
Preparation of the reaction mixture for transglutamination
using wild type hGH and 1.3-diaminol propanol.

| | Wild type hGH | | | | | TrisHCl | Total |
|---|---|---|---|---|---|---|---|
| | working solution | | 1,3-dap | mTGase | H₂O | pH 8.0 | vol. |
| Stock sol. | 4.0 mg/ml | H₂O | 1 M | Varies | | 1 M | |
| Reaction | 320 μl | 280 μl | 90 μl | 10 μl | 290 μl | 10 μl | 1 ml |
| Final conc. | ≈60 μM | | 90 mM | 0.2-0.3 μM | | 10 mM | |
| | (1.28 mg/ml) | | | (10-15 μg/ml) | | | |

The hGH working solution was first prepared from its stock solution which is in TrisHCl, 5 mM, pH 7.0 and then used for the reaction.
*1,3-dap: 1,3-diamino-propanol

CE Analysis

The frozen sample from the transglutamination reaction was first diluted 1:10 with H₂O and CE was carried out using P/ACE MDQ from Beckman Coulter with a capillary of 30.5 cm×50 um i.d., UV detection was performed at 214 nm at 20° C. Since the pl of transamincated hGH was about 5.80-6.20, the CE analysis was run in TrisHCl, 50 mM, pH 8.0.

The capillary was first conditioned with 0.1 M HCl for 0.5 m, rinsed with distilled water for 1.5 m, injected sample for 0.5 m, and finally run at +15 kV for 25 m for sample separation.

From the CE profiles, the retention time for wild type hGH, mono-substituted hGH at Q141 and mono-substituted hGH at Q40 were 6.5, 7.9 and 10 m, respectively.

### Example 5

Evaluation of High Selective mTGase Mutants

The improvement of the selectivity of the mutants was compared with that from the wild type mTGase (in AlaPro-mTGase form) from S. mobaraensis. The selectivity of the N-terminal variants was evaluated by the Screening Assay. The selectivity of all the mutants were evaluated by CE analysis on the transglutamination reactions using wild type hGH as substrate and 1.3-diamino propanol as the amine donor.

### Example 6

Effect of Different N-Terminal Sequences to the Selectivity of mTGase from S. ladakanum

Variants of the mTGase from S. ladakanum with different N-terminal extra sequences were compared for the selectivity at hGHQ141 (using hGHQ40N as substrate) over hGHQ40 (using hGHQ141 as the substrate) using the assay described in Example 3.

Results shown in Table 2 indicated that the overall selectivity of the mTGase from S. ladakanum is higher than that of AlaPro-mTGase from S. mobaraensis.

Among the 4 different versions of mTGase from S. ladakanum, which had different N-terminal sequence, GlyPro-mTGase stands out to have the highest selectivity with a RS of 2.7. Although the crystal structure of the mTGase from S. ladakanum is not available, the result shown in Table 2 indicated that the N-terminus of mTGase may also involved in the conformation change of binding pocket of mTGase to its substrate, e.g. hGH. The improved selectivity may be due to the squeezing down of the binding pocket of mTGase, which makes the Gln residue at certain site of substrate e.g. Q141 of hGH, to be more preferable for mTGase catalyzed transglutamination. The selectivity of wild type GlyPro-mTGase from S. ladakanum was further confirmed by transglutamination reaction measured by CE. Based on the results above, further mutations were generated on the GlyPro-mTGase of S. ladakanum.

Since no difference in selectivity for different N-terminal versions of mTGase from S. mobaraensis was observed, the AlaPro-mTGase from S. mobaraensis was used as the reference and the improvement of selectivity was evaluated by RS (relative selectivity) calculated from the Screening assay described in Example 3.

TABLE 2
Comparison of the selectivity of variants of mTGase from
S. ladakanum having different N-terminal sequences.

| | | Activity towards | Activity towards | | |
|---|---|---|---|---|---|
| | | hGHQ40N | hGHQ40N | Selectivity | |
| Source of mTGase | mTGase variant | (RFU/sec/μg) | (RFU/sec/μg) | (Q141/Q40) | RS¹ |
| S. mobaraensis² | mTGase | 3.35 | 0.98 | 3.4 | 1.0 |
| | Met-mTGase | 5.44 | 1.24 | 4.4 | 1.3 |
| | AlaPro-mTGase | 5.05 | 1.56 | 3.2 | 1.0 |
| | GlyPro-mTGase | 4.28 | 1.13 | 3.8 | 1.2 |
| S. ladakanum | Mature mTGase | 3.55 | 0.63 | 5.6 | 1.7 |
| | Met-mTGase | 4.74 | 0.80 | 6.0 | 1.8 |
| | AlaPro-mTGase | 6.91 | 1.02 | 6.8 | 2.1 |
| | GlyPro-mTGase | 4.25 | 0.48 | 8.8 | 2.7 |

The AlaPro-mTGase from S. mobaraensis was used as reference. The selectivity calculated was the activity towards hGHQ141 (using hGHQ40N as substrate) over hGH40 (using hGHQ141 as the substrate).
¹RS: Relative selectivity, the ratio of the selectivity of the mutant versus that of the wild type mTGase from S. mobaraensis.

### Example 7

mTGase Mutants Generated by Site-Directed Mutation Based on GlyPro-mTGase from S. ladakanum

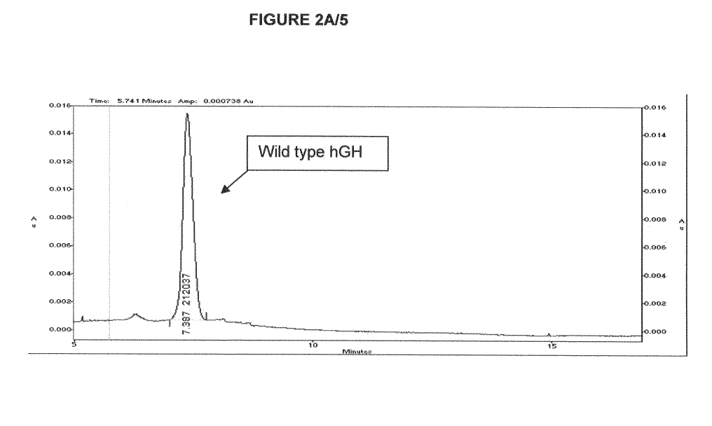

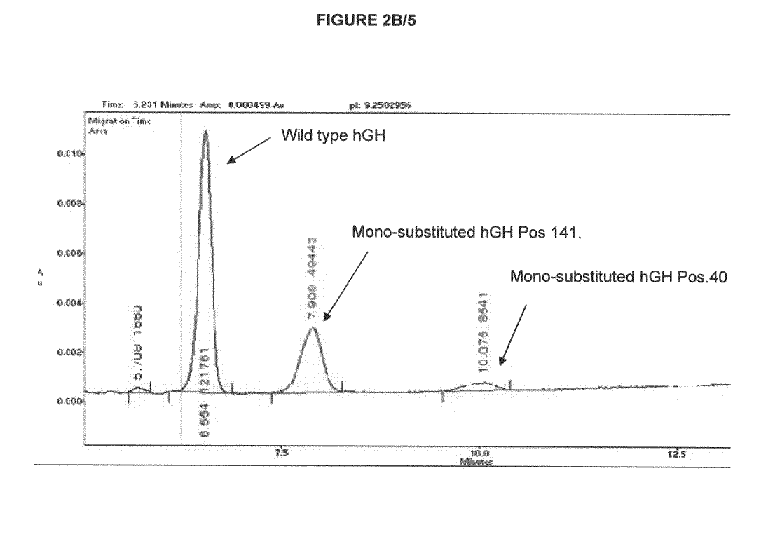

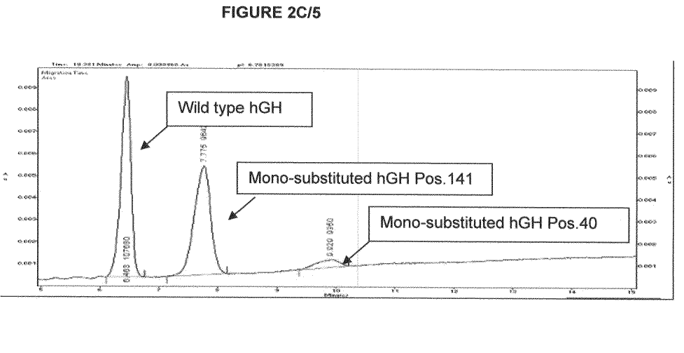

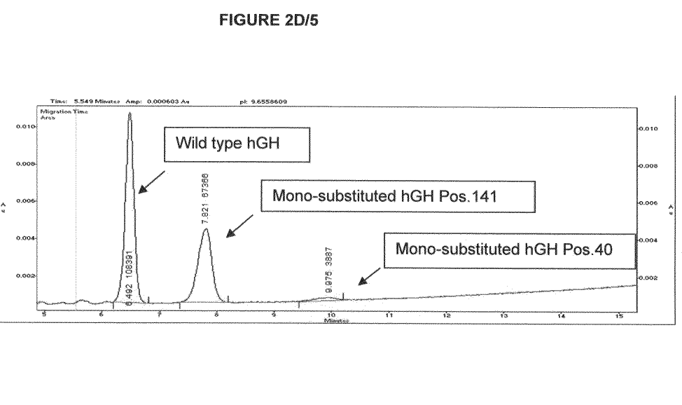

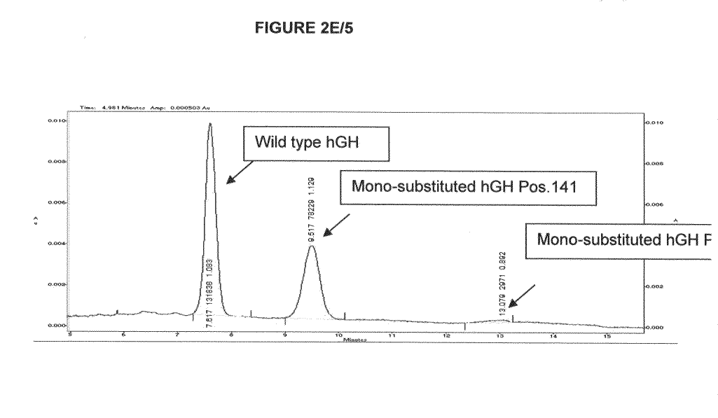

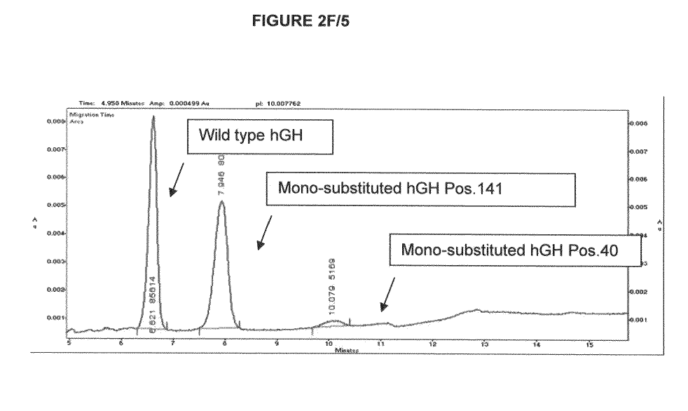

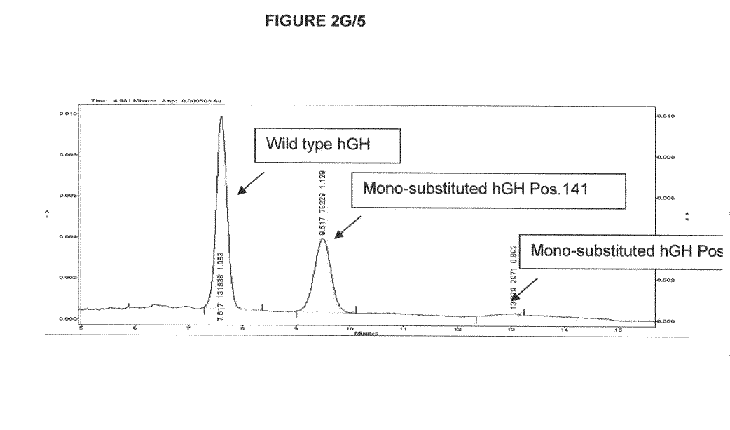

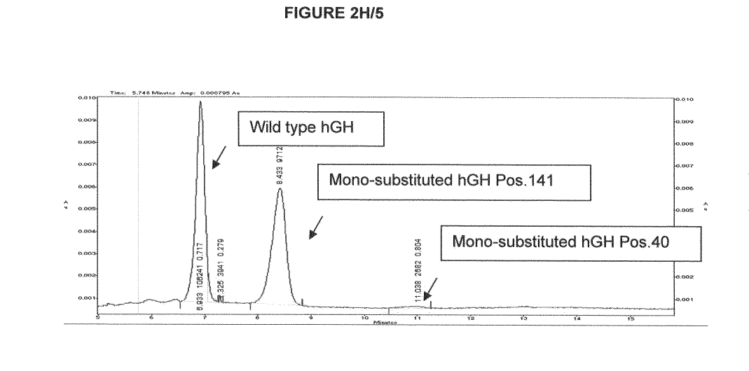

Transglutamination reactions were performed using the GlyPro-mTGase with wild type hGH as the substrate and 1.3-diamino propanol as the amine donor. The selectivity for transglutamination at Q141 of hGH over Q40 was evaluated by CE. The improvement of the selectivity was evaluated using the AlaPro-mTGase from S. mobaraensis as the reference and GlyPro-mTGase from S. ladakanum as the benchmark. The results are listed in Table 3. The CE graphs for each mutant are shown in FIG. 2B to FIG. 2H.

The results listed in Table 3 shows that all the mutants including Y75A, Y75F, Y75N, Y62H_Y75N and Y62H_Y75F had improved selectivity than that of the GlyPro-mTGase-SL. The highest selective mutant is GlyPro-mTGase_Y62H_Y75F with a selectivity of 36.2 when the hGH conversion rate is 49.2%, which is 6.4 times higher than that of AlaPro-mTGase from S. mobaraensis. Further measurements were performed with 7.6 times improvement of selectivity under lower hGH conversion rate of 38.1%. Repeated transglutamination reaction using GlyPro-mTGase_Y62H_Y75F variant gave a 7.6 times higher improvement in selectivity than that of AlaPro-mTGase from S. mobaraensis when the hGH conversion rate for both the mutant and reference mTGase were about 40%.

TABLE 3
Comparison of selectivity of mTGase variants

| | | | | | | |
|---|---|---|---|---|---|---|
| | | hGH | Reaction | | | |
| | Selectivity | conv. | time | Enzyme Conc. | | |
| mTGase variant | (Q141/Q40) | rate (%) | (m) | used (μg/ml) | RS¹ | CE figure¹ |

S. mobaraensis²

| | | | | | | |
|---|---|---|---|---|---|---|
| AlaPro-mTGase | 5.7 | 33 | 30 | 9.6 | 1.0 | FIG. 2B |

S. ladakanum

| | | | | | | |
|---|---|---|---|---|---|---|
| GlyPro-mTGase | 10.3 | 55 | 15 | 16 | 1.8 | FIG. 2C |
| GlyPro- | 17.3 | 40 | 300 | 17.7 | 3.0 | FIG. 2D |
| mTGase_Y75A | | | | | | |
| GlyPro- | 29.1 | 41 | 60 | 12.9 | 5.1 | FIG. 2E |
| mTGase_Y75F | | | | | | |
| GlyPro- | 19.3 | 44 | 120 | 6.5 | 3.4 | FIG. 2F |
| mTGase_Y75N | | | | | | |
| GlyPro- | 26.3 | 38 | 75 | 34.5 | 4.6 | FIG. 2G |
| mTGase_Y62H_Y75N | | | | | | |
| GlyPro- | 36.2 | 49.4 | 120 | 7 | 6.4 | FIG. 2H |
| mTGase_Y62H_Y75F | | | | | | |
| | 76.5² | 38.1 | 60 | 8.8 | 7.6 | (Separate |
| | (Ref. = 10.1) | | | | | exp²) |
| | Ref. 10.1 | 39 | 45 | 4.6 | 1 | |

¹From the CE profiles, the retention time for wild type hGH, mono-substituted hGH at Q141 and mono-substituted hGH at Q40 were 6.5, 7.9 and 10 m, respectively.
²This experiment was performed separately where the reference, AlaPro-mTGase, had a selectivity of 10.1, and the hGH conversion rate was 38.1.

The sequence of GlyPro-mTGase_Y62H_Y75F from S. ladakanum is given as SEQ ID No. 4.

The sequence of the peptide Propeptide-(3C)-MTGase from S. ladakanum is given in SEQ ID No. 5.

### Example 8

Testing of mTGase from S. ladakanum and S. mobarense for their Selectivity Towards Gln-141 vs. Gln-40 in hGH

This assay uses two hGH mutants each having an asparagine residue instead of a glutamine at one of positions Gln-40 and Gln-141, leaving only one glutamine to react. The preparation of said mutants are described in Kunkel T A et al., Methods in Enzymology 154, 367-382 (1987), and Chung Nan Chang et al., Cell 55, 189-196 (1987). The hGH mutant Q40N is a model substrate for Gln-141 in hGH, and Q141N is a model substrate for Gln-40.

To 400 μl of buffer solution with 225 mM 1,3-diamino-2-propanol and 35 mM Tris (pH has been adjusted to 8.0 by addition of concentrated HCl), 600 μl of mutant hGH (1.5 mg/ml) and 5 μl of TGase (1.6 mg/ml) are added, The reaction mixture is incubated for 30 minutes at 25° C.

The subsequent analysis is performed by FPLC using a Mono Q 5/5 GL 1 ml (GE Health) column and UV detection at 280 nm. Buffer A: 20 mM triethanolamine pH 8.5; Buffer B: 20 mM triethanolamine 0.2 M NaCl pH 8.5; flow rate: 0.8 ml/min. The elution gradient is defined as following:

| Step | Time/min | % A | % B |
|---|---|---|---|
| 1 | 2.00 | 100.0 | 0.0 |
| 2 | 4.00 | 70.0 | 30.0 |
| 3 | 5.00 | 70.0 | 30.0 |
| 4 | 35.00 | 50.0 | 50.0 |

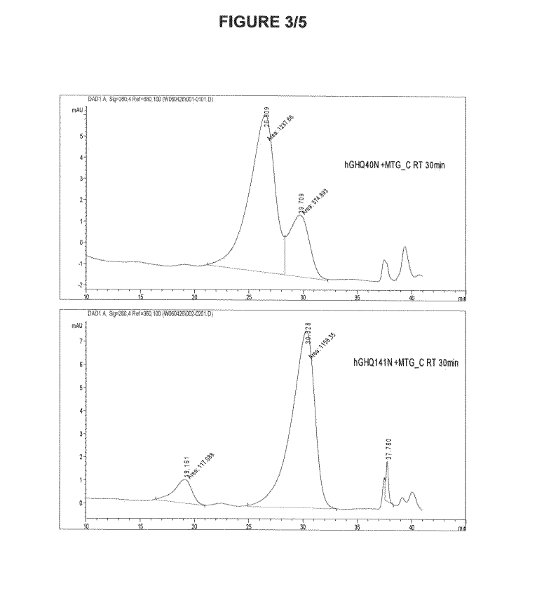

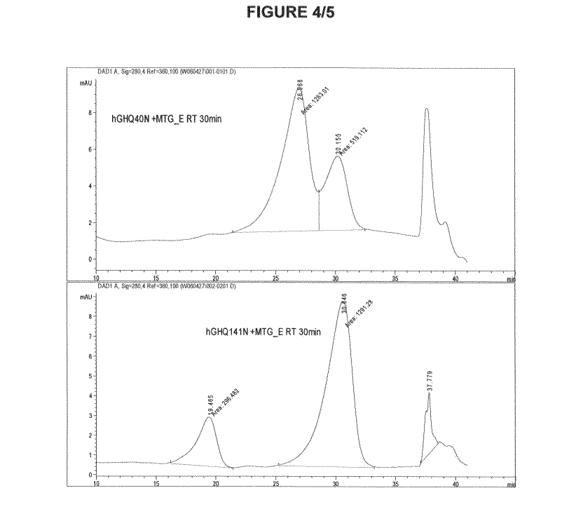

The selectivity ratio is then calculated from the ratio of the two areas (in arbitrary units) under the curves (shown in FIGS. 3 and 4) attributed to the two products, Q141 and Q40. The result achieved when using TGase from S. ladakanum (SEQ ID No. 1) and S. mobarense (SEQ ID No. 2) is shown in Table 4. Q40N+its product-Q141=Q141N+its product-Q40, and are normalized to 100.

TABLE 4

| | | product- | | product- | Transamination | Gln 40 vs. Gln 141 |
|---|---|---|---|---|---|---|
| Enzyme | Q40N | Q141 | Q141N | Q40 | Gln 40 vs. Gln 141 | (normalized) |
| mTGase from | 29 | 71 | 81 | 19 | 19:71 | 21:79 |
| S. mobarense | | | | | | |
| mTGase from | 23 | 77 | 90 | 10 | 10:77 | 11:89 |
| S. ladakanum | | | | | | |

### Example 9

### PEGylation of hGH

a) hGH is dissolved in phosphate buffer (50 mM, pH 8.0). This solution is mixed with a solution of amine donor, e.g. 1,3-diamino-propan-2-ol dissolved in phosphate buffer (50 mM, 1 ml, pH 8.0, pH adjusted to 8.0 with dilute hydrochloric acid after dissolution of the amine donor).
Finally a solution of TGase (˜40 U) dissolved in phosphate buffer (50 mM, pH 8.0, 1 ml) is added and the volume is adjusted to 10 ml by addition of phosphate buffer (50 mM, pH 8). The combined mixture is incubated for approximately 4 hours at 37° C. The temperature is lowered to room temperature and N-ethyl-maleimide (TGase inhibitor) is added to a final concentration of 1 mM. After further 1 hour the mixture is diluted with 10 volumes of tris buffer (50 mM, pH 8.5).
b) The transaminated hGH obtained from a) may then optionally be further reacted to activate a latent functional group if present in the amine donor.
c) The functionalised hGH obtained from a) or b) is then reacted with a suitably functionalised PEG capable of reacting with the functional group introduced into hGH. As an example, an oxime bond may be formed by reacting a carbonyl moiety (aldehyde or ketone) with an alkoxyamine.

### Example 10

### PEGylation of hGH

Step a

hGH is dissolved in triethanol amine buffer (20 mM, pH 8.5, 40% v/v ethylene glycol). This solution is mixed with a solution of amine donor, e.g. 1,3-diamino-propan-2-ol dissolved in triethanol amine buffer (20 mM, pH 8.5, 40% v/v ethylene glycol, pH adjusted to 8.6 with dilute hydrochloric acid after dissolution of the amine donor).

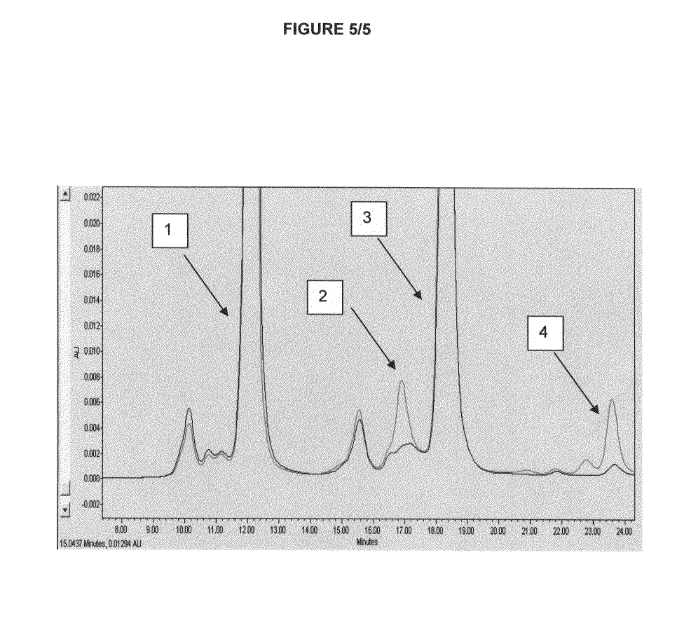

Finally a solution of AlaPro-mTGase from S. mobarense (AlaPro-mTGase-SM) or GlyPro-mTGase Y62H_Y75F from S. ladakanum (GlyPro-mTGase Y62H_Y75F-SL) (˜0.5-7 mg/g hGH) dissolved in 20 mM PB, pH 6.0 is added and the volume is adjusted to reach 5-15 mg/ml hGH (20 mM, pH 8.5). The combined mixtures are incubated for 1-25 hours at room temperature. The reaction mixture is analysed by CIE HPLC as shown in Table 5 and FIG. 5. TA 40 means transaminated in position 40, TA 141 means transaminated in position 141, and TA 40/141 means transaminated in position 40 and 141.

TABLE 5

| .Reaction time | hGH left | TA 40 | TA 141 | TA 40/141 |
|---|---|---|---|---|
| (hrs)/enzyme | (area %) | (area %) | (area %) | (area %) |
| 1/AlaPro-mTGase-SM | 63.6 | 4.4 | 27.5 | 1.3 |
| 1/GlyPro-mTGase | 63.0 | 1.7 | 32.0 | 0.3 |
| Y62H_Y75F-SL | | | | |
| 22/AlaPro-mTGase-SM | 38.4 | 6.2 | 40.5 | 3.6 |
| 22/GlyPro-mTGase | 48.3 | 3.5 | 37.5 | 0.6 |
| Y62H_Y75F-SL | | | | |
| 25/GlyPro-mTGase | 9.9* | 2.5 | 65 | 3.7 |
| Y62H_Y75F-SL* | | | | |

*75% hGH in starting material

Step b

The transaminated hGH obtained from step a) may then optionally be further reacted to activate a latent functional group if present in the amine donor.

Step c

The functionalised hGH obtained from step a) or b) is then reacted with a suitably functionalised PEG capable of reacting with the functional group introduced into hGH. As an example, an oxime bond may be formed by reacting a carbonyl moiety (aldehyde or ketone) with an alkoxyamine.

### Example 11

Selectivity of TGase Mutants of S. ladakanum

Each reaction was carried out at room temperature in a 20 mM Tris-HCl, pH 7.4 and 200 mM NaCl buffer containing 100 μM monodansyl cadaverine (which was prepared by dissolving the powder with acetic acid and buffered with 1 M Tris-HCl, pH 8.5) and 50 μM Q141N or Q40N human growth hormone. The TGase was added to the mixture to start reactions. Fluorescence was measured at ext/em. 340/520 nm every 30 seconds. The initial reaction rates for Q40N and Q141N were estimated and used to calculate the selectivity.

The results of this experiment for several S. ladakanum GlyPro-TGase mutants are shown in Table 6.

TABLE 6

| Specific mutation | RSA Q141 | RSA Q40 | RS |
|---|---|---|---|
| GlyPro-S250A | 4.23 | 2.57 | 1.65 |
| GlyPro-S250C | 5.34 | 3.02 | 1.77 |
| GlyPro-S250D | 1.04 | 0.76 | 1.37 |
| GlyPro-S250F | 2.63 | 1.85 | 1.42 |
| GlyPro-S250G | 2.42 | 1.77 | 1.37 |
| GlyPro-S250H | 2.25 | 1.40 | 1.61 |
| GlyPro-S250L | 3.59 | 1.90 | 1.89 |
| GlyPro-S250M | 3.25 | 2.22 | 1.46 |
| GlyPro-S250P | 3.99 | 1.86 | 2.15 |
| GlyPro-S250Q | 1.92 | 1.62 | 1.18 |
| GlyPro-S250R | 0.83 | 0.59 | 1.41 |
| GlyPro-S250V | 0.96 | 0.51 | 1.88 |
| GlyPro-S250W | 2.43 | 1.30 | 1.87 |
| GlyPro-S250Y | 1.71 | 0.95 | 1.81 |
| GlyPro-Y62L | 0.20 | 0.07 | 2.92 |
| GlyPro-Y62M | 0.28 | 0.10 | 2.79 |
| GlyPro-Y62N | 0.18 | 0.05 | 3.60 |
| GlyPro-Y62T | 0.19 | 0.10 | 2.00 |
| GlyPro-Y75C | 0.18 | 0.06 | 2.77 |
| GlyPro-Y75L | 0.18 | 0.06 | 2.57 |
| GlyPro-Y75M | 0.18 | 0.10 | 1.61 |
| GlyPro-Y75A | 0.06 | 0.03 | 2.45 |

RSAQ141: specific activity towards Q141-hGH relative to that of the wild type TGase
RSAQ40: specific activity towards Q40-hGH relative to that of the wild type TGase
RS: Relative selectivity, the ratio of the selectivity of the mutant versus that of the wild type TGase

1. A transglutaminase peptide having a specificity for Gln-141 of hGH compared to Gln-40 of hGH, which is higher than the specificity of a peptide having an amino acid sequence as shown in SEQ ID No. 1 for Gln-141 of hGH compared to Gln-40 of hGH.

2. A transglutaminase peptide according to claim 1 comprising an amino acid sequence having at least 80% identity with the amino acid sequence in SEQ ID No. 1.

3. A transglutaminase peptide according to claim 2, wherein said amino acid sequence is modified in one or more of the positions corresponding to the amino acid residues Tyr62, Tyr75 and Ser250 of SEQ ID No. 1.

4. An isolated peptide comprising an amino acid sequence having at least 80% identity with the amino acid sequence in SEQ ID No. 1, wherein said sequence is modified in one or more of the positions to the amino acid residues Tyr62, Tyr75 and Ser250 of SEQ ID No. 1.

5. An isolated peptide according to claim 4 comprising an amino acid sequence as defined in SEQ ID No. 1, wherein said sequence is modified in one or more of the positions corresponding to the amino acid residues Tyr62, Tyr75 and Ser250 of SEQ ID No. 1.

6. An isolated peptide according to claim 5, wherein the amino acid residue in the position corresponding to Tyr62 is Met, Asn, Thr, or Leu.

7. An isolated peptide according to claim 3, wherein the amino acid residue in the position corresponding to Tyr75 is Ala, Met, Leu, or Cys.

8. An isolated peptide according to claim 3, the amino acid residue in the position corresponding to Ser250 is Cys, Leu, Pro, Trp, Tyr, or Val.

9. An isolated peptide comprising an amino acid sequence having at least 80% identity with the amino acid sequence in SEQ ID No. 1, wherein said sequence is modified by the addition of from one to ten amino acid residues in the N-terminal.

10. An isolated peptide according to claim 9 comprising an amino acid sequence as defined in SEQ ID No. 1, wherein said sequence is modified by the addition of from one to ten amino acids in the N-terminal.

11. An isolated peptide comprising an amino acid sequence having at least 80% identity with the amino acid sequence in SEQ ID No. 2, wherein said sequence is modified by the addition of from one to ten amino acids in the N-terminal.

12. An isolated peptide according to claim 11 comprising an amino acid sequence as defined in SEQ ID No. 2, wherein said sequence is modified by the addition of from one to ten amino acids in the N-terminal.

13. An isolated peptide according to claim 9, wherein said sequence is modified by the addition of two amino acids in the N-terminal.

14. An isolated peptide according to claim 4, which peptide has a specificity for Gln-141 of hGH compared to Gln-40 of hGH, which is higher than the specificity of a peptide having an amino acid sequence as shown in SEQ ID No. 2 for Gln-141 of hGH compared to Gln-40 of hGH.

15. An isolated peptide comprising the sequence of SEQ ID No. 1.

16. An isolated peptide having the sequence of SEQ ID No. 1.

17. A nucleic acid construct encoding a peptide according to claim 1.

18. A vector comprising the nucleic acid construct of claim 17.

19. A host cell comprising the vector of claim 18.

20. A composition comprising a peptide according to claim 1.

21. A method for conjugating a peptide, wherein said method comprises reacting said peptide with an amine donor in the presence of a peptide according to claim 1.

22. A method according to claim 21, wherein said growth hormone is hGH or a variant or derivative thereof.

23. A method for conjugating a growth hormone according to claim 22, wherein the amount of growth hormone conjugated at the position corresponding to position Gln-141 of hGH as compared to the amount of hGH conjugated at the position corresponding to position Gln-40 of hGH is significantly increased in comparison with the amount of hGH conjugated at the position corresponding to position Gin-141 of hGH as compared to the amount of hGH conjugated at the position corresponding to position Gln-40, when a peptide having the amino acid sequence as shown in SEQ ID No.2 is used in said method.

24. A method for the preparation of a hGH conjugated at the position corresponding to position 141, wherein said method comprises reacting said hGH with an amine donor in the presence of a peptide according to claim 1.

25. A method for the preparation of a pharmaceutical composition of a conjugated growth hormone, which method comprises a step of reacting said hGH or variant or derivative thereof with an amine donor in the presence of a peptide according to claim 1.

26. A method for the preparation of a pharmaceutical composition of a pegylated growth hormone, which method comprises a step of reacting said hGH or variant or derivative thereof with an amine donor in the presence of a peptide according to claim 1, and using the resulting conjugated growth hormone peptide for the preparation of a pegylated growth hormone, wherein said pegylation takes place at the conjugated position.

27. (canceled)

28. A method for treatment of a disease or disorder related to lack of growth hormone in a patient, which method comprises administration of a pharmaceutical preparation as prepared by use of a method according to claim 25 to a patient in need thereof.

29. A method for treatment of a disease or disorder related to lack of growth hormone in a patient, which method comprises administration of a pharmaceutical preparation as prepared by use of a method according to claim 26 to a patient in need thereof.


SEQUENCE LISTING (5 sequences)

SEQ ID NO: 1 - protein, 331 aa, Streptoverticillium ladakanum
DSDERVTPPAEPLDRMPDPYRPSYGRAETIVNNYIRKWQQVYSHRDGRKQQMTEEQREWLSYGCVGVTWVNSGQYPTNRLAFAFFDEDKYKNELKNGRPRSGETRAEFEGRVAKDSFDEAKGFQRARDVASVMNKALENAHDEGAYLDNLKKELANGNDALRNEDARSPFYSALRNTPSFKDRNGGNHDPSKMKAVIYSKHFWSGQDRSGSSDKRKYGDPEAFRPDRGTGLVDMSRDRNIPRSPTSPGESFVNFDYGWFGAQTEADADKTVWTHGNHYHAPNGSLGAMHVYESKFRNWSDGYSDFDRGAYVVTFVPKSWNTAPDKVKQGWP

SEQ ID NO: 2 - protein, 331 aa, Streptomyces mobaraensis
DSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWP

SEQ ID NO: 3 - protein, 380 aa, Artificial
  TGase from Streptoverticillium ladakanum as expressed in E.coli
GSGSGSGTGEEKRSYAETHRLTADDVDDINALNESAPAASSAGPSFRAPDSDERVTPPAEPLDRMPDPYRPSYGRAETIVNNYIRKWQQVYSHRDGRKQQMTEEQREWLSYGCVGVTWVNSGQYPTNRLAFAFFDEDKYKNELKNGRPRSGETRAEFEGRVAKDSFDEAKGFQRARDVASVMNKALENAHDEGAYLDNLKKELANGNDALRNEDARSPFYSALRNTPSFKDRNGGNHDPSKMKAVIYSKHFWSGQDRSGSSDKRKYGDPEAFRPDRGTGLVDMSRDRNIPRSPTSPGESFVNFDYGWFGAQTEADADKTVWTHGNHYHAPNGSLGAMHVYESKFRNWSDGYSDFDRGAYVVTFVPKSWNTAPDKVTQGWP

SEQ ID NO: 4 - protein, 333 aa, Artificial
  GlyPro-mTGase_Y62H_Y75F from S. ladakanum
GPDSDERVTPPAEPLDRMPDPYRPSYGRAETIVNNYIRKWQQVYSHRDGRKQQMTEEQREWLSHGCVGVTWVNSGQFPTNRLAFAFFDEDKYKNELKNGRPRSGETRAEFEGRVAKDSFDEAKGFQRARDVASVMNKALENAHDEGAYLDNLKKELANGNDALRNEDARSPFYSALRNTPSFKDRNGGNHDPSKMKAVIYSKHFWSGQDRSGSSDKRKYGDPEAFRPDRGTGLVDMSRDRNIPRSPTSPGESFVNFDYGWFGAQTEADADKTVWTHGNHYHAPNGSLGAMHVYESKFRNWSDGYSDFDRGAYVVTFVPKSWNTAPDKVTQGWP

SEQ ID NO: 5 - protein, 388 aa, artificial
  propeptide-(3C)-MTGase from S. ladakanum
GSGSGSGTGEEKRSYAETHRLTADDVDDINALNESAPAASSAGPSFRAPLEVLFQGPDSDERVTPPAEPLDRMPDPYRPSYGRAETIVNNYIRKWQQVYSHRDGRKQQMTEEQREWLSHGCVGVTWVNSGQFPTNRLAFAFFDEDKYKNELKNGRPRSGETRAEFEGRVAKDSFDEAKGFQRARDVASVMNKALENAHDEGAYLDNLKKELANGNDALRNEDARSPFYSALRNTPSFKDRNGGNHDPSKMKAVIYSKHFWSGQDRSGSSDKRKYGDPEAFRPDRGTGLVDMSRDRNIPRSPTSPGESFVNFDYGWFGAQTEADADKTVWTHGNHYHAPNGSLGAMHVYESKFRNWSDGYSDFDRGAYVVTFVPKSWNTAPDKVTQGWP